SEQUENCE LISTING (12 sequences)

SEQ ID NO: 1 - DNA, 18 bp, Artificial Sequence
  Synthetic Construct
ccgggctttgatctttgc

SEQ ID NO: 2 - DNA, 18 bp, Artificial Sequence
  Sythetic Construct
gcaaagatcaaagcccgg

SEQ ID NO: 3 - DNA, 18 bp, Artificial Sequence
  Sythetic Construct
ccgggtcagttcttttgc

SEQ ID NO: 4 - DNA, 18 bp, Artificial Sequence
  Sythetic Construct
gcaaaagaactgacccgg

SEQ ID NO: 5 - DNA, 24 bp, Artificial Sequence
  Sythetic Construct
atgaccacagtccatgccatcact

SEQ ID NO: 6 - DNA, 24 bp, Artificial Sequence
  Sythetic Construct
tgttgaagtcgcaggagacaacct

SEQ ID NO: 7 - DNA, 20 bp, Artificial Sequence
  Sythetic Construct
tggatcctcgaacctggcta

SEQ ID NO: 8 - DNA, 20 bp, Artificial Sequence
  Sythetic Construct
ctcaggctgcaaagtctcca

SEQ ID NO: 9 - DNA, 20 bp, Artificial Sequence
  Sythetic Construct
ccccatcgacttccgcgacg

SEQ ID NO: 10 - DNA, 20 bp, Artificial Sequence
  Sythetic Construct
tgggtgcggtgctgctgatg

SEQ ID NO: 11 - DNA, 20 bp, Artificial Sequence
  Sythetic Construct
cgtcggtgctgctgctcaca

SEQ ID NO: 12 - DNA, 20 bp, Artificial Sequence
  Sythetic Construct
cggccaccaccaggaagcag

(19) United States
(12) Patent Application Publication
(10) Pub. No.: US 2019/0365705 A1
(43) Pub. Date: Dec. 5, 2019

(54) TOPICAL TOCOTRIENOL COMPOSITIONS AND METHODS OF INCREASING SKIN STEM CELLS

(71) Applicant: OHIO STATE INNOVATION FOUNDATION, Columbus, OH (US)

(72) Inventors: Chandan SEN, Upper Arlington, OH (US); Savita KHANNA, Columbus, OH (US); Subhadip GHATAK, Columbus, OH (US)

(21) Appl. No.: 16/479,467

(22) PCT Filed: Jan. 22, 2018
(86) PCT No.: PCT/US2018/014681

§ 371 Date: Jul. 19, 2019

Related U.S. Application Data
(60) Provisional application No. 62/448,647, filed on Jan. 20, 2017.
(60) Provisional application No. 62/485,043, filed on Apr. 13, 2017.

(51) Int. Cl. A61K 31/355; A61K 9/00; A61K 47/44

(52) U.S. Cl. CPC A61K 31/355; A61K 45/06; A61K 47/44; A61K 9/0014

(57) ABSTRACT

The present disclosure relates to topical tocotrienol compositions, dosing regimens, and methods for increasing the stem cell content of the skin. The present invention further relates to topical tocotrienol compositions, dosing regimens, and methods for the treatment of skin disorders.

### CROSS REFERENCE TO RELATED APPLICATIONS

This application claims the benefit of U.S. Provisional Patent Application Ser. No. 62/448,647 filed Jan. 20, 2017 and U.S. Provisional Patent Application Ser. No. 62/485,043 filed Apr. 13, 2017, the disclosures of which are expressly incorporated herein by reference.

### FIELD

The present disclosure relates to topical tocotrienol compositions, dosing regimens, and methods for increasing the stem cell content of the skin. The present disclosure further relates to topical tocotrienol compositions, dosing regimens, and methods for the treatment of skin disorders.

### BACKGROUND

In mammals, the skin provides a protective barrier impermeable to harmful microbes and also prevents dehydration. To perform its functions while being confronted with the physicochemical traumas of the environment, the skin tissue undergoes continual rejuvenation through homeostasis, and, in addition, is primed to undergo wound repair in response to injury. While a number of compositions are currently used to treat an array of skin injuries and skin disorders, improved treatments are needed that can repair and rejuvenate the skin.

The compounds, compositions, and methods disclosed herein address these and other needs.

### SUMMARY

Disclosed herein are topical tocotrienol compositions, dosing regimens, and methods for increasing the stem cell content of the skin. The present invention further comprises topical tocotrienol compositions, dosing regimens, and methods for the treatment of skin disorders.

In one aspect, disclosed herein is a method of increasing skin stem cells in a subject, comprising:

administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and delta-tocotrienol; and
a pharmaceutically acceptable excipient;
   wherein the tocotrienol composition is administered at a dose of about 1 mg/cm² to about 10 mg/cm²; and
   wherein the tocotrienol composition is administered in an amount sufficient to increase skin stem cells in the subject.

In one embodiment, the increase in skin stem cells is determined by measuring the expression of a stem cell marker. In one embodiment, the stem cell marker is selected from Oct4, Sox9, K17, K15, or LGR-6. In one embodiment, the stem cell marker is selected from Oct4, Sox9 or LGR-6.

In another aspect, disclosed herein is a method for the prevention or treatment of a skin disorder, comprising:

administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and delta-tocotrienol; and
a pharmaceutically acceptable excipient;
   wherein the tocotrienol composition is administered at a dose of about 1 mg/cm² to about 10 mg/cm²; and
   wherein the tocotrienol composition is administered in an amount sufficient to prevent or treat the skin disorder in the subject.

In one embodiment, the skin disorder is selected from skin injury, UV exposure related injury, sunburn, melanoma, wrinkles, psoriasis, severe dryness, itchiness, microbial infections, or fungal infections

In one embodiment, the tocotrienol composition further comprises an additional dermatological agent. In one embodiment, the additional dermatological agent is an antibiotic or antifungal.

In one embodiment, the tocotrienol composition further comprises alpha-tocopherol. In one embodiment, the tocotrienol composition comprises tocopherol, by weight percent of total, less than a percent selected from the group consisting of: 50%; 40%; 30%; 20%; 15%; 10%; 5%; and 1%. In one embodiment, the tocotrienol composition is substantially free of tocopherol.

In one embodiment, the tocotrienol composition is derived from palm oil.

### BRIEF DESCRIPTION OF THE DRAWINGS

The accompanying figures, which are incorporated in and constitute a part of this specification, illustrate several aspects described below.

FIGS. 1A-1C. Induction of epidermal anagen hair cycling in skin. (FIG. 1A) Photomicrographs of mice dorsal skin at day 7 and day 21 showing induction of anagen hair cycling in TRF treated shaved skin. (FIG. 1B) Dermascopic images from inset (FIG. 1A) of the dorsal mouse skin at day 7 and day 21. TRF induced anagen hair on day 21. (FIG. 1C) Photomicrograph of formalin fixed paraffin embedded H&E stained sections showing more anagen hair follicles with dermal papillae reaching subcutaneous fat layer in TRF treated sections at day 21. Scale bar=500 μm. Inset: close-up of a hair follicle with outer root sheath (ORS), inner root sheath (IRS), cortex (C) and medulla (M). Hair follicles were quantified from H&E stained sections. Data are mean±SD. (n=6)*p<0.001.

FIGS. 2A-2C. Developmental hair folliculogenesis. (FIG. 2A) LGR6 and CD34 immunostaining counterstained with nuclear DAPI. Upper panel: LGR6 and CD34 in fetal skin. Scale=50 μm. Bottom panel: Adult skin. Scale=20 μm. Epidermal and dermal junction marked by white dashed lines. Fluorescence plotted as relative florescence unit (RFU). Data are mean±SD. (n=3)† p<0.01. (FIG. 2B) H&E stained section showing development of hair follicle from the fetal skin epidermis. Scale=50 μm. (FIG. 2C) TRF treatment on adult skin showed morphological characteristics similar to murine fetal skin. Schematic diagram shown on the left-hand panel while actual region was marked in the H&E stained sections with dashed lines. Scale bar=50 μm. The number of “hair germ” and “hair peg” were quantified from H&E stained sections and expressed graphically. (n=6) nd, not detected.

FIGS. 3A-3C. Keratinocyte proliferation in hair folliculogenesis. (FIG. 3A) IVIS image from repTOP™mitoIRE showing cell proliferation in animal treated with TRF or placebo (PBO) on days 7 and 21. (FIG. 3B) Immunohistochemical localization of Ki67 in paraffin sections of mice skin showing abundance of Ki67⁺ cells in mice treated (days 7 and 21) with TRF compared to PBO. Scale bar=50 μm. Ki67⁺ cells were quantified and plotted graphically. Data are mean±SD. (n=3) § p<0.05; †, p<0.01. (FIG. 3C) Photomicrograph of H&E stained paraffin sections showing epidermal thickening in mice treated (days 7 and 21) with TRF. Scale bar=50 μm. Epidermal thickness was quantified and plotted graphically. Data are mean±SD. (n=4)*p<0.001.

FIGS. 4A-4C. Epidermal junctional proteins in murine skin folliculogenesis. (FIG. 4A) TRF lowered (day 21) the expression of Claudin, ZO-2 and E-cadherin in murine skin. Sections were counter stained with DAPI. Dermal-epidermal junction is indicated by dashed white line. Scale bar=50 μm. Abundance of junctional proteins in FIG. 4A were quantified and expressed graphically as mean±SD. (n=6). § p<0.05; †, p<0.01; *p<0.001. (FIG. 4B) Transepidermal water loss (TEWL) was measured from the dorsal skin of mice after topical application of TRF or PBO for 21 days. Data are mean±SD. (n=6) †, p<0.01. (FIG. 4C) Keratinocytes (HaCaT cells) treated with pure tocotrienol (1 μM, 24 h) showed lower expression of E-cadherin in the cell membrane, and increased nuclear translocation of β-catenin. Scale=20 μm.

FIGS. 5A-5D. Induction of β-Catenin and nuclear translocation. (FIG. 5A) TRF induced (day 21) β-catenin in murine epidermis. Counterstained with DAPI. Scale bar=50 μm. Fluorescence intensity was plotted graphically. Data are mean±SD. (n=3) § p<0.05. (FIG. 5B) Confocal microscopy showing TRF-induced translocation (day 21) of β-catenin into the nucleus. Counterstained with DAPI. Dermal (der) and epidermal (epi) junction indicated by white dashed line. Scale bar=10 μm. (FIG. 5C) Proximity ligation assay (PLA) showed β-catenin-Tcf3 co-localization in the nucleus in TRF treated adult skin. Scale bar=20 μm. (FIG. 5D) Robust expression of SOX9, OCT4, K15 and K17 in TRF treated (day 21) adult skin epidermis. Scale bar=20 μm.

FIGS. 6A-6D. β-catenin-Tcf3 interaction in induction of pluripotency. (FIG. 6A) Design of FAM labelled TCF decoy and eight-base mismatch scramble control. (FIG. 6B) Theoretical scheme of Tcf3 decoy strategy. i, Normal β-catenin signaling. β-catenin/Tcf3 complex binding facilitates gene expression. ii. β-catenin may preferentially bind to the Tcf3 decoy region, which competitively inhibits target gene activation. (FIG. 6C) Expression of SOX9, OCT4, K17 and K15 in Tcf3 scramble (0.1 μM) and decoy (0.1 μM) transfected cells after pure tocotrienol treatment (1 μM, 24 h). Scale bar=50 μm. (FIG. 6D) The number of positive cells per field were quantified and plotted graphically. Data are mean±SD. (n=3) § p<0.05; †, p<0.01.

FIGS. 7A-7C. β-Catenin inhibition arrested inducible anagen hair cycling. (FIG. 7A) IWR-1, a β-catenin inhibitor, significantly attenuated TRF-induced epidermal β-catenin expression in the adult skin. Counterstained with DAPI. Scale bar=500 μm. β-Catenin signal was quantified and plotted graphically. Data are mean±SD. (n=3, †, p<0.01). The dermal (der) and epidermal (epi) junction is marked by a dashed line in the inset picture. Scale bar=20 μm. (FIG. 7B) IWR-1 inhibited TRF-induced translocation of β-catenin to the nucleus. Scale bar=50 μm. (FIG. 7C) Proposed schematic diagram of TRF-induced hair folliculogenesis.

FIGS. 8A-8D. TRF induces hair follicles. (FIG. 8A) Representative photomicrograph of formalin fixed paraffin embedded H&E stained sections showing larger number of anagen hair follicle in TRF treated (days 7 and 14) sections. Scale bar=500 μm. Total number of hair follicles were quantified from the H&E stained sections from each week and plotted graphically. Data are mean±SD. (n=6)*p<0.001. (FIG. 8B) Digital photomicrographs of C57BL/6 (wt) and db/db mice. db/db mice showing less hair growth in naired skin on day 14. (FIG. 8C) Application of TRF induced (day 21) hair growth in dorsal skin of diabetic mouse. (FIG. 8D) Representative photomicrograph of formalin fixed paraffin embedded H&E stained sections showing large number of anagen hair follicle in TRF-treated (day 21) db/db skin. Scale bar=500 μm. The number of anagen hair follicles on day 21 were quantified from H&E stained sections and plotted graphically. Data are mean±SD. (n=6) § p<0.05.

FIGS. 9A-9B. Occludin, ZO-1, and β-catenin expression in the epidermis treated with TRF. (FIG. 9A) Representative photograph of formalin fixed paraffin embedded adult skin sections showing no change (day 21) in occludin and ZO-1 expression in the epidermis treated with TRF. Counterstained with DAPI. The dermal and epidermal junction is indicated by a dashed line. Scale bar=20 μm. (FIG. 9B) HaCaT keratinocytes treated with TRF (equivalent to 1 μM tocotrienol) showed increased nuclear translocation of β-catenin compared to PBO at 24 h. Co-localization is shown in white. Scale=50 μm.

FIGS. 10A-10J. Gene expression in skin. (FIG. 10A) β-catenin is abundantly expressed in fetal skin (E18.5). Confocal microscopic image showing translocation of β-catenin into the nucleus. Counterstained with DAPI. Dermal and epidermal junction is indicated by dashed line in the left panel. Scale bar=200 μm. Co-localization is shown in white in the right panel. Scale bar=20 μm. (FIG. 10B) β-catenin expression in the adult skin epidermis counterstained with DAPI at day 21 post-treatment with TRF or PBO. Scale bar=200 μm. (FIG. 10C) E-cadherin knockdown in HaCaT keratinocytes induced nuclear translocation of β-catenin. White dots mark co-localization. Scale bar=20 μm. (FIG. 10D) Proximity ligation assay (PLA) with both + and − probe and + probe alone validating the assay for which data is shown in FIG. 5C. (FIG. 10E) (i) Proximity ligation assay (PLA) showing the interaction of β-catenin and Tcf3 in the nucleus 24 h after treatment with 1 μM pure tocotrienol. (ii) PLA showing the β-catenin and Tcf3 co-localization as dots (related to FIG. 10Ei. (iii) PLA with (+) probe and (−) probe alone validating the assay. Scale bar=50 μm (FIG. 10F) The nuclear lysates from HaCaT keratinocytes after 24 h treatment with either vehicle or 1 μM pure tocotrienol were subjected to immunoprecipitation with β-catenin antibody. The immunoprecipitates (IP) were subjected to SDS-PAGE and subjected to immunoblotting (IB) for the detection of Tcf3. (FIG. 10G) KLF4, c-MYC, and NANOG expression in adult skin epidermis on day 21 post-treatment with PBO and TRF. Dermal and epidermal junction is indicated by black dashed line. Scale bar=20 μm. (FIG. 10H) Laser captured epidermis was subjected to (FIG. 10I) quantitative PCR analysis of Sox9, Oct4 and Lgr6 (day 21 after PBO and TRF treatment). Data are mean±SD, † p<0.01 compared to PBO. (FIG. 10J) SOX9, OCT4, K15 and K17 expression in adult skin epidermis on day 7 post-treatment with TRF. Dermal and epidermal junction is indicated by a dashed line. Scale bar=20 μm.

FIGS. 11A-11C. KLF4, c-MYC, and NANOG expression after tocotrienol treatment. (FIG. 11A) Expression of KLF4, c-MYC, and NANOG in Tcf3 scramble and decoy transfected cells after pure tocotrienol treatment (1 μM, 24 h). Scale bar=50 μm. (FIG. 11B) The number of positive cells per field were quantified and plotted graphically. Data are mean±SD. (n=3) § p<0.05. nd, not detected. (FIG. 11C) Schematic representation of how IWR-1 is inhibiting β-catenin nuclear translocation and subsequent activation of target gene.

FIGS. 12A-12C. Topical IWR-1 studies. (FIG. 12A) Design of topical IWR-1 studies. (FIG. 12B) H&E stained skin sections showing that IWR-1 attenuated TRF-induced increase in the number of hair follicles at day 11 (day 7 post TRF application). Scale bar=50 μm. Hair follicles were enumerated and expressed graphically as mean±SD, *p<0.001 compared to vehicle TRF with vehicle. (FIG. 12C) Graphical representation of Ki67⁺ cells in formalin fixed paraffin embedded skin sections of mice treated with IWR-1 or vehicle DMSO followed by TRF treatment. IWR-1 treatment blunted TRF-induced cell proliferation in the epidermis. Scale bar=50 μm

FIG. 13. Specificity of the antibodies used in the study was validated using no antibody control and isotype controls of respective host species. The white dash line indicates the epidermal and dermal junctions. Scale bar=50 μm.

### DETAILED DESCRIPTION

Disclosed herein are topical tocotrienol compositions, dosing regimens, and methods for increasing the stem cell content of the skin. The present invention further comprises topical tocotrienol compositions, dosing regimens, and methods for the treatment of skin disorders.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition (e.g., skin disorder). The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human. In some embodiments, the pharmacokinetic profiles of the systems of the present invention are similar for male and female subjects.

As used herein, the term “controlled-release” or “controlled-release drug delivery” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “about” or “approximately” as used herein when referring to a measurable value such as an amount, a percentage, and the like, is meant to encompass variations of ±20%, ±10%, ±5%, or ±1% from the measurable value.

### Methods and Therapeutic Regimens

In one aspect, disclosed herein is a method of increasing skin stem cells in a subject, comprising:

administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and delta-tocotrienol; and
a pharmaceutically acceptable excipient;
   wherein the tocotrienol composition is administered at a dose of about 1 mg/cm² to about 10 mg/cm²; and
   wherein the tocotrienol composition is administered in an amount sufficient to increase skin stem cells in the subject.

In one embodiment the tocotrienol composition is administered at a dose of about 1 mg/cm² to about 10 mg/cm²; about 2 mg/cm² to about 9 mg/cm²; about 3 mg/cm² to about 8 mg/cm²; about 4 mg/cm² to about 7 mg/cm²; or about 5 mg/cm² to about 6 mg/cm².

In one embodiment, the tocotrienol composition is administered at a dose of about 1 mg/cm², about 2 mg/cm², about 3 mg/cm², about 4 mg/cm², about 5 mg/cm², about 6 mg/cm², about 7 mg/cm², about 8 mg/cm², about 9 mg/cm², or about 10 mg/cm². In a particular embodiment, the tocotrienol composition is administered at a dose of about 5 mg/cm².

In one embodiment, the tocotrienol composition is administered at a volume of about 0.2 ml/cm². In one embodiment, the tocotrienol composition is administered at a volume of about 0.05 ml/cm² to about 1 ml/cm² to about.

In one embodiment, the increase in skin stem cells is determined by measuring the expression of a stem cell marker. In one embodiment, the stem cell marker is selected from Oct4, Sox9, K17, K15, or LGR-6. In one embodiment, the stem cell marker is selected from Oct4, Sox9 or LGR-6. In some embodiments, any stem-ness marker that is a marker of stem cells can be used in the methods disclosed herein.

The increase in skin stem cells in the subject may be in comparison to skin stem cells in the subject prior to administration of the topical tocotrienol composition. The increase in skin stem cells in the subject may be in comparison to skin stem cells (for example, absolute numbers or relative percentages of skin stem cells) in another subject (or population of subjects) that does not receive administration of the topical tocotrienol composition (for example, receive a placebo, or a composition containing the corresponding pharmaceutically acceptable excipient without the tocotrienols).

In one embodiment, the tocotrienol composition further comprises an additional dermatological agent. In one embodiment, the additional dermatological agent is an antibiotic or antifungal.

In one embodiment, the tocotrienol composition further comprises alpha-tocopherol. In one embodiment, the tocotrienol composition comprises tocopherol, by weight percent of total, less than a percent selected from the group consisting of: 50%; 40%; 30%; 20% 15%; 10%; 5%; and 1%. In one embodiment, the tocotrienol composition is substantially free of tocopherol.

In one embodiment, the tocotrienol composition is derived from palm oil.

In another aspect, disclosed herein is a method for the prevention or treatment of a skin disorder, comprising:

administering to the subject an effective amount of a topical tocotrienol composition three times per week, wherein the topical tocotrienol composition comprises:
at least one tocotrienol selected from the group consisting of: alpha.-tocotrienol; beta-tocotrienol; gamma-tocotrienol; and delta-tocotrienol; and
a pharmaceutically acceptable excipient;
   wherein the tocotrienol composition is administered at a dose of about 1 mg/cm² to about 10 mg/cm²; and
   wherein the tocotrienol composition is administered in an amount sufficient to prevent or treat the skin disorder in the subject.

In one embodiment, the tocotrienol composition further comprises an additional dermatological agent. In one embodiment, the additional dermatological agent is an antibiotic or antifungal.

In one embodiment, the tocotrienol composition further comprises alpha-tocopherol. In one embodiment, the tocotrienol composition comprises tocopherol, by weight percent of total, less than a percent selected from the group consisting of: 50%; 40%; 30%; 20%; 15%; 10%; 5%; and 1%. In one embodiment, the tocotrienol composition is substantially free of tocopherol.

In one embodiment, the tocotrienol composition is derived from palm oil.

In one embodiment, the skin disorder is selected from skin injury, UV exposure related injury, sunburn, melanoma, wrinkles, psoriasis, severe dryness, itchiness, microbial infections, or fungal infections.

Topical administration of a tocotrienol rich fraction of a Tocovid Suprabio® capsule enriched the stem cell biomarkers found within the skin and increased the stern cell content. In certain embodiments, the tocotrienol rich fraction is administered three times per week, with each dosage comprising a dose of tocotrienols of 5 mg/cm². In certain embodiments, the tocotrienol rich fraction is administered three times per week, with each dosage comprising a dose of tocotrienols of 800 ul/8 cm².

The increase in stem content of the skin has numerous applications. This disclosure describes, for example, dosing regimens using topical tocotrienol creams that may be used in wound healing, for example, treating burn wounds. In addition, this tocotrienol composition (TCT) can also be effective in repairing and rejuvenating adult skin in wrinkle management, aging, UV exposure related injury, sunburn, melanoma, and other skin related skin disorders (for example, psoriasis, severe dryness, itchiness, stretch marks, acne, and microbial/fungal diseases). Psoriasis is a persistent skin disorder presenting inflammation with redness and thickened areas often on the scalp, elbows, knees, and/or lower back. Severe skin dryness and itching can be caused by loss of lipids and inflammation. Stretch marks are scar-like manifestations visible on skin that has changed surface area due to skin stretching. In one embodiment, the skin disorder to be treated or prevented is selected from skin injury, UV exposure related injury, sunburn, melanoma, wrinkles, psoriasis, severe dryness, itchiness, microbial infections, and/or fungal infections.

Additional skin disorders can include, for example, dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

In some embodiments, the dosing regimens disclosed herein can reduce the severity of a burn injury and associated scar formation. The process of wound healing after a traumatic injury or surgery, is a complex process progressing from blood clotting, inflammation and intrusion of immune system white cells, migration of immature connective tissue cells into the region, laying down of a connective tissue matrix, closure of the wound, and scarification or regeneration of a normal tissue structure.

The skin harbors its own progenitor cells to ensure tissue renewal in the absence of injury, and the hair follicles contain multipotent stern cells that helps in regeneration and repair of the epidermis following wounding.

In one embodiment, disclosed herein is a method for treating wrinkles. The tocotrienols can reduce wrinkles and thus signs of aging on the skin. The tocotrienol rich fraction (TCT) can reduce the look of expression lines and wrinkles and to make the skin look younger. In one embodiment, the tocotrienol composition can be used to treat hair loss.

In another aspect, provided herein is a method for activating the proliferation of new skin stem cells, comprising:

administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and del ta-tocotri enol ; and
a pharmaceutically acceptable excipient;
   wherein the tocotrienol composition is administered at a dose of about 1 mg/cm² to about 1 mg/cm²; and
   wherein the tocotrienol composition is administered in an amount sufficient to activate the proliferation of new skin stem cells in the subject.

### Tocotrienols

The natural vitamin E family is composed of eight members, equally divided into two classes; tocopherols (TCP) and tocotrienols (TCT or TE). TCP are characterized by a saturated phytyl side chain with three chiral carbons whereas TCTs possess a famesyl side chain with double bonds at carbons 3, 7, and 11. Within each class, isomers are differentiated by α, β, γ, and δ according to the position and degree of methylation on the chromanol head. TCPs represent the primary form of vitamin E in green leafy vegetables, while TCTs are found in highest concentration in seeds of monocotyledons that include the wheat, rice, barley, and palm.

In some embodiments, the tocotrienol in the tocotrienol compositions can comprise at least one of the following:

alpha-tocotrienol, beta-tocotrienol, gamma-tocotrienol, and/or delta-tocotrienol.

In one embodiment, at least one tocotrienol is selected from the group consisting of: alpha-tocotrienol; beta-tocotrienol; gamma-tocotrienol; and delta-tocotrienol.

In some embodiments, provided are such methods, wherein the tocotrienol is administered topically, in a formulation comprising approximately 17-34% alpha-tocotrienol, approximately 2-4% beta-tocotrienol, approximately 27-54% gamma-tocotrienol, approximately 8-23% delta tocotrienol, and approximately 14-32% alpha tocopherol, by weight of those five ingredients.

In some embodiments, provided are such methods, wherein the tocotrienol is administered topically, in a formulation comprising approximately 25% alpha-tocotrienol, approximately 3% beta-tocotrienol, approximately 37% gamma-tocotrienol, approximately 15% delta tocotrienol, and approximately 20% alpha tocopherol, by weight of those five ingredients.

In some embodiments, provided are such methods, wherein the tocotrienol is administered topically, in a formulation comprising 17-34% alpha-tocotrienol, 2-4% beta-tocotrienol, 27-54% gamma-tocotrienol, 8-23% delta tocotrienol, and 14-32% alpha tocopherol, by weight of those five ingredients.

In some embodiments, provided are such methods, wherein the tocotrienol is administered topically, in a formulation comprising 25% alpha-tocotrienol, 3% beta-tocotrienol, 37% gamma-tocotrienol, 15% delta tocotrienol, and 20% alpha tocopherol, by weight of those five ingredients.

In some embodiments, provided are such methods, wherein the tocotrienol is administered topically, in a formulation having approximately 24-30% alpha-tocotrienol, 2-6% beta-tocotrienol, approximately 35-46% gamma-tocotrienol, approximately 14-20% delta tocotrienol, and approximately 19-26% alpha tocopherol, by weight of those five ingredients.

In some embodiments, provided are such methods, wherein the tocotrienol is administered topically, in a formulation having approximately 12-15% alpha-tocotrienol, 1-3% beta-tocotrienol, approximately 17.5-23% gamma-tocotrienol, approximately 7-10% delta tocotrienol, and approximately 9.5-13% alpha tocopherol, by weight of each ingredient in a 50:50 composition containing 50% active compounds.

In some embodiments, provided are such methods, wherein the tocotrienol is administered topically, in a formulation having approximately 25% alpha-tocotrienol, 4% beta-tocotrienol, approximately 36% gamma-tocotrienol, approximately 15% delta tocotrienol, and approximately 20% alpha tocopherol, by weight of those five ingredients.

In some embodiments, provided are such methods, wherein the tocotrienol is administered topically, in a formulation having approximately 15-30% alpha-tocotrienol, approximately 30-50% gamma-tocotrienol, approximately 2-15% delta tocotrienol, and approximately 20-30% alpha tocopherol, by weight of those four ingredients.

In some embodiments, provided are such methods, wherein the tocotrienol is administered topically, in a formulation having approximately 23% alpha-tocotrienol, approximately 41% gamma-tocotrienol, approximately 9% delta tocotrienol, and approximately 25% alpha tocopherol, by weight of those four ingredients.

In further embodiments, provided are such methods, wherein the tocotrienol composition is derived from palm oil.

In some embodiments, provided are such methods, wherein the tocotrienol composition is Tocovid SupraBio®. Tocovid SupraBio is a softgel capsule which contains Tocomin, a natural extract of palm phytonutrients consisting of mixed-tocotrienols, tocopherols, mixed carotenoids, phytosterols and squalene. Mixed-tocotrienols are one of the key ingredients in Tocovid SupraBio and they are a more potent form of Vitamin E compared with tocopherol.

In some embodiments, the tocotrienol composition comprises approximately 6-12% alpha-tocotrienol, approximately 0.7-1.1% beta-tocotrienol, approximately 8-16% gamma-tocotrienol, approximately 3-7% delta tocotrienol, and approximately 4-10% alpha tocopherol.

In some embodiments, the tocotrienol composition comprises approximately 7.5% alpha-tocotrienol, approximately 0.9% beta-tocotrienol, approximately 11% gamma-tocotrienol, approximately 4.5% delta tocotrienol, and approximately 6% alpha tocopherol.

In some embodiments, the tocotrienol composition comprises 6-12% alpha-tocotrienol, 0.7-1.1% beta-tocotrienol, 8-16% gamma-tocotrienol, 3-7% delta tocotrienol, and 4-10% alpha tocopherol.

In some embodiments, the tocotrienol composition comprises 7.5% alpha-tocotrienol, 0.9% beta-tocotrienol, 11% gamma-tocotrienol, 4.5% delta tocotrienol, and 6% alpha tocopherol.

Also provided are such methods, wherein the tocotrienol composition administered comprises tocopherol, by weight percent total, less than a percent selected from the group consisting of: 50%; 40%; 30%; 20%; 15%; 10%; 5%; and 1%.

In some embodiments, the tocotrienol composition in 1 ml of solution comprises: Tocomin 50%—200 mg; Soya Oil—305.4 mg; Labrasol—50 mg; and Cremophor—50 mg.

Tocomin 50% typically comprises: d-Alpha-Tocotrienol: 61.52 mg {6.15% (w/v) of total capsule}; d-Gamma-Tocotrienol: 112.80 mg {11.28% (w/v) of total capsule}; d-Delta-Tocotrienol: 25.68 mg {2.57% (w/v) of total capsule}; d-Alpha-Tocopherol 91.60 IU; Plant Squalene 51.28 mg; Phytosterol Complex 20.48 mg; and Phytocarotenoid Complex 360.00 μg.

In some embodiments, tocotrienol rich fraction (TRF) was obtained from commercially available Tocovid Suprabio capsules (Patel, V, Rink, C, Gordillo, G M, Khanna, S, Gnyawali, U, Roy, S, et al. (2012). Oral tocotrienols are transported to human tissues and delay the progression of the model for end-stage liver disease score in patients. J Nutr 142: 513-519). In some embodiments, each capsule contains Tocomin 50% which typically provides d-Alpha-Tocotrienol 61.52 mg, d-Gamma-Tocotrienol 112.80 mg, d-Delta-Tocotrienol 25.68 mg, d-Alpha-Tocopherol 91.60 IU, Plant Squalene 51.28 mg, Phytosterol Complex 20.48 mg, Phytocarotenoid Complex 360.00 ug. In some embodiments, each capsule contains 144.86 mM of d-Alpha-Tocotrienol. In the placebo, the tocotrienol was replaced by soya oil. For in vitro experiments, pure tocotrienol was provided by Excelvite Inc, New Jersey.

### Topical Compositions

In some embodiments, a topical cream of 15% tocotrienol concentration is used in the methods disclosed herein (available and marketed by Hovid Bhd (Malaysia)).

### Formula

Methyl paraben 0.1%

Propyl paraben 0.01%

Carbomer 0.3%

Glycerin 4.0%

Triethanolaime 0.6%

Cetomacrogol emulsifying wax 5%

Cetyl alcohol 1.5%

Stearic acid 1.5%

EVNol 50% (containing 50% tocotrienols and tocopherol): 30% (containing 15% tocotrienols/tocopherol final concentration)

Propylene Glycol Caprylate 5%

Modified Corn Starch 1.5%

Carotenoids 20%: 0.1%

Water qs 100

EVNol 50% contains a minimum of 50% vitamin E whereby approximately 10% is alpha-tocopherol, 12% of alpha-tocotrienols, 18.5% of gamma-tocotrienols, 2% of beta-tocotrienols and 7.5% of delta-tocotrienols. EVNol 50% is produced by ExcelVite Sdn Bhd Malaysia.

In some embodiments, a topical cream of 0.5% tocotrienol concentration is used in the methods disclosed herein (available and marketed by Hovid Bhd (Malaysia)).

### Formula

Methyl paraben 0.1%

Propyl paraben 0.01%

Carbomer 0.3%

Triethanolaime 0.9%

Polyoxyl 40 Castor Oil 1%

Cetyl alcohol 2%

Stearic acid 4%

EVNol 50%: 1% (containing approximately 0.5% tocotrienols/tocopherol final concentration)

Liquid paraffin 3.5%

Propylene glycol 5%

Titanium dioxide 0.5%

Water qs 100

In some embodiments, a topical cream of 1% tocotrienol concentration is used in the methods disclosed herein (available and marketed by Hovid Bhd (Malaysia)).

### Formula

Methyl paraben 0.1%

Propyl paraben 0.01%

Carbomer 0.3%

Triethanolaime 0.9%

Polyoxyl 40 Castor Oil 1%

Cetyl alcohol 2%

Stearic acid 4%

EVNol 50%: 2% (containing approximately 1% tocotrienols/tocopherol final concentration)

Liquid paraffin 3.5%

Propylene glycol 5%

Titanium dioxide 0.5%

Water qs 100

In some embodiments, the tocotrienol composition may contain at least one of the following:

Methyl paraben and propyl paraben—functions as preservatives. The Cosmetic Ingredient Review (CIR) reviewed the safety of methylparaben, propylparaben, and butylparaben in 1984 and concluded they were safe for use in cosmetic products at levels up to 25%. In December 2005, the CIR Panel again determined that there was no need to change its original conclusion that parabens are safe as used in cosmetics. They are included in the FDA Inactive Ingredients Guide for topical preparations. It is also accepted for use as a food additive in Europe and affirmed GRAS Direct Food Substances in USA at levels up to 0.1% (Handbook of Pharmaceutical Excipients, 4th Edition, 2003, published by the Pharmaceutical Press and the American Pharmaceutical Association);
Carbomer—gelling agent for the aqueous phase. Included in the FDA Inactive Ingredients Guide (oral suspension and tablets, ophthalmic, rectal and topical preparations). It is used extensively in nonparenteral products, particularly topical liquid and semisolid preparations. Carbomer is generally regarded as essentially nontoxic and nonirritant material; there is no evidence in humans of hypersensitivity reactions to carbomer used topically (Handbook of Pharmaceutical Excipients, 4th Edition, 2003, published by the Pharmaceutical Press and the American Pharmaceutical Association);
Glycerin—also referred to as glycerol. This functions as a humectant. Glycerin is listed as GRAS and accepted as food additives. It is included in the FDA :Inactive :Ingredients Guide for topical preparations;
Triethanolamine—an alkalizing agent to neutralize the carbomer to build up the gel structure as well as an emulsifying agent. Also referred to as Trolamine in USPNF. Included in the FDA Inactive Ingredients Guide (rectal, topical and vaginal preparations);
Polyoxyl 40 castor oil—an emulsifying agent. Included in the FDA Inactive ingredients Guide (IV injections and ophthalmic solutions);

Cetomacrogol emulsifying wax—also referred to nonionic emulsifying wax. It is used as an emulsifying agent and stiffening agent. Cetomacrogol emulsifying wax is included in the FDA inactive ingredient guides (topical aerosols, emulsions, lotions and ointments). It is generally regarded as essentially nontoxic and nonirritant material;

Cetyl alcohol—an emulsifying agent and stiffening agent. Included in the FDA inactive Ingredient Guides (creams, emulsions and topical aerosols);
Stearic acid is a stiffening agent. It is listed as GRAS and accepted as a food additive in Europe (fatty acids). Included in the FDA Inactive Ingredient Guides (topical and vaginal preparations) Propylene Glycol Caprylate is an emulsifying agent;
Dry Flo AF—hydrophobically modified corn starches which is used for its ability to enhance the aesthetics of a skin care products and mitigate greasiness in the cream formulation. It is manufactured by National Starch and Chemical Company, New Jersey, USA.

In one embodiment, the tocotrienol composition (or the EVNol 50%) contains approximately 10% of alpha-tocopherol, 12% of alpha-tocotrienols, 20.6% of gamma-tocotrienols, 1.5% of beta-tocotrienols and 5% of delta-tocotrienols.

In one embodiment, the tocotrienol composition is administered in a controlled-release pharmaceutical dosage form. In one aspect, the controlled-release pharmaceutical dosage form comprises a biodegradable controlled-release polymer.

Compositions, as described herein, comprising an active compound (tocotrienols) and an excipient of some sort may be useful in a variety of applications. For example, pharmaceutical compositions comprising an active compound and an excipient may be useful for the treatment or prevention of a skin disorders, or for the repair and rejuvenation of skin due to increased stem cell content in the skin.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methyl cellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, b enzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropyl cellulose (MHPC), carboxymethyl cellulose (CMC) and its various salts, including, e.g., the sodium salt, hydroxyethylcarboxymethyl cellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl -sn-glycero-3-Phosphoethanolamine-N-[Methoxy(Polyethylene glycol)-2000], and 1,2-Distearoyl -sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [ Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

### Combination Therapies

The tocotrienol compositions have been shown here to have beneficial effects on the stem cell content of the skin. Thus, in some embodiments, the tocotrienol compositions disclosed herein can be administered in combination with an additional dermatological agent. Additional dermatological agents can be selected, for example, from the group consisting of antibiotics, antifungals, and/or corticosteroids. In one embodiment, the tocotrienol composition is administered in combination with an antibiotic or antifungal.

The antibiotic can be selected, for example, from the group consisting of penicillin, penicillin G, penicillin V, procaine, benzathine, cloxacillin, dicloxacillin, methicillin, nafcillin, oxacillin, azlocillin, carbenicillin, piperacillin, piperacillin plus tazobactam, ticarcillin, mezlocillin, cefadroxil, cefazolin, cephalexin, cephalothin, cephapirin, cephradine, cefaclor, cefamandole, cefmetazole, cefonicid cefotetan, cefoxitin, cefprozil, cefuroxime, loracarbef, cefepime, cefixime, cefoperazone, cefotaxime, cefpodoxime, ceftazidime, ceftibuten ceftizoxime, ceftriaxone, Imipenem, meropenem, aztreonam, clavulanic acid, sulbactam, tazobactam, ampicillin, ampicillin plus sulbactam, amoxycillin, amoxicillin, amoxicillin plus clavulanate potassium, bacampicillin, clavulanic acid plus amoxycillin, aztreonam, imipenem, streptomycin, kanamycin, neomycin, gentamycin, tobramycin, amikacin, netilmicin, gentamicin, vancomycin, clindamycin, azithromycin, clarithromycin, clindamycin, roxithromycin, dirithromycin, spiramycin, josamycin, erythromycin, lincomycin, bacitracin, colistin, polymyxin B, bacitracin, amphotericin, nystatin, rifampicin, tetracycline, chlortetracycline, oxytetracycline, demeclocycline, minocycline, doxycycline, chloramphenicol, ciprofloxacin, enoxacin, grepafloxacin, levofloxacin, lomefloxacin, norfloxacin, ofloxacin, sparfloxacin, trovafloxacin, cinoxacin, nalidixic acid, clindamycin, linezolid, spectinomycin, quinupristin, dalfopristin, trimethoprim, trimethoprim-sulfamethoxazole, sulfanilamide, sulfadiazine, sulfamethoxazole, sulfisoxazole, sulfamethizole, silver sulfadiazine, mafenide, and mixtures thereof.

The antifungal can be selected, for example, from the group consisting of clotrimazole, griseofulvin, undecylenic, econazole, miconazole, ketaconazole, sulconazole, oxiconazole, fluconazole, itraconazole, nystatin, naftifine, terbinafine, ciclopirox, butenafine, haloprogin, tolnaftate, and mixtures thereof.

The corticosteroid can be selected, for example, from the group consisting of hydrocortisone, prednisone, fluprednisolone, dexamethasone, betamethasone, betamethasone valerate, methylprednisolone, fluocinolone acetonide, flurandrenolone acetonide, fluorometholone, cortisone, prednisolone, alclometasone, amcinonide, betamethasone, clobetasol, clocortolone, desonide, desoximetasone, diflorasone, fluocinonide, flurandrenolide, fluticasone, halcinonide, halobetasol, mometasone, flumethasone, prednicarbate, triamcinolone, and mixtures thereof.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### Epidermal E-Cadherin Dependent P-Catenin Pathway is Phytochemical-Inducible and Accelerates Anagen Hair Cycling

Unlike the epidermis, which regenerates continually, hair follicles anchored in the subcutis periodically regenerate by spontaneous repetitive cycles of growth (anagen), degeneration (catagen) and rest (telogen). The loss of hair follicles in response to injuries or pathologies such as alopecia endangers certain inherent functions of the skin. Thus, it is of interest to understand mechanisms underlying follicular regeneration in adults. In this example, a phytochemical rich in the natural vitamin E tocotrienol (TRF; or sometimes referred to as TCT) served as a productive tool to unveil a novel epidermal pathway of hair follicular regeneration. Topical TRF application markedly induced epidermal hair follicle development akin to that during fetal skin development. This was observed in the skin of healthy as well as diabetic mice which are known to be resistant to anagen hair cycling. TRF suppressed epidermal E-cadherin followed by 4-fold induction of β-catenin and its nuclear translocation. Nuclear β-catenin interacted with Tcf3. Such sequestration of Tcf3 from its otherwise known function to repress pluripotent factors induced the plasticity factors Oct4, Sox9, Klf4, c-Myc and Nanog. Pharmacological inhibition of β-catenin arrested anagen hair cycling by TRF. This example reports epidermal E-cadherin/β-catenin as a novel pathway capable of inducing developmental folliculogenesis in the adult skin, and reports the use of tocotrienol compositions in methods for increasing the stem cell content of the skin and for the treatment of skin disorders.

### Background

Mammalian hair follicles harbor a multipotent stem cell niche of diverse developmental origin that continuously self-renew, differentiate, regulate hair growth, and contribute to skin homeostasis. Hair follicle neogenesis may also replenish the stem cell pool for skin rejuvenation and regenerative healing. Post-natal hair follicles regenerate periodically by spontaneous cycle of growth (anagen), apoptosis-driven regression (catagen), and relative quiescence (telogen). Developmentally, hair follicle morphogenesis takes place during the late embryonic and early neonatal period. It begins with the formation of a small cluster of epithelial placode that is marked by the expression of cytokeratin 17 and Lgr6. However, de novo formation of hair follicles is typically not observed in adults except during wound-induced activation of the epidermal Wnt/β-catenin pathway.

Previous works recognized tocotrienol as a potent neuroprotective agent. Compared to the widely known form of vitamin E tocopherol, tocotrienol has similar phenolic head. However, instead of saturated phytyl tail in tocopherol, tocotrienol has an isoprenoid tail. During studies testing the effect of tocotrienol on peripheral neuropathy, an unexpected observation was noticed that murine skin topically treated with tocotrienol showed more robust hair growth. Although there is anecdotal evidence reported in the literature claiming improvement of hair growth in humans by tocotrienol, the underlying mechanisms remain elusive.

### Results

Induction of Anagen Hair Cycling in Skin

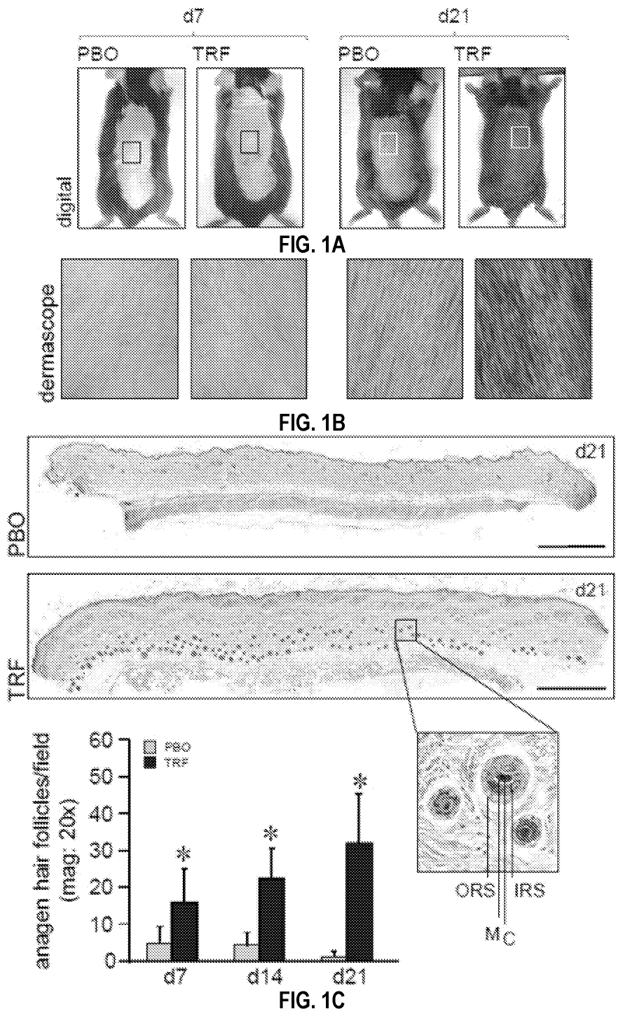

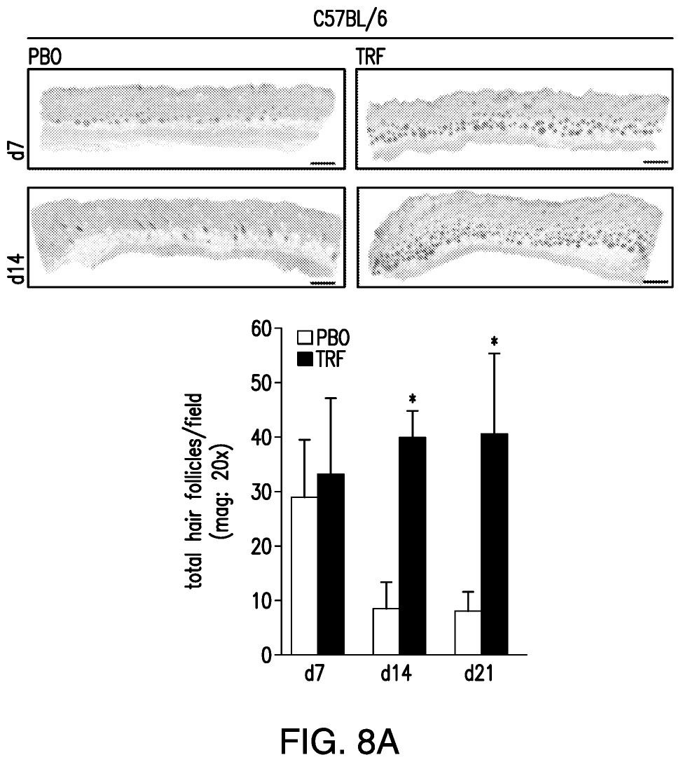

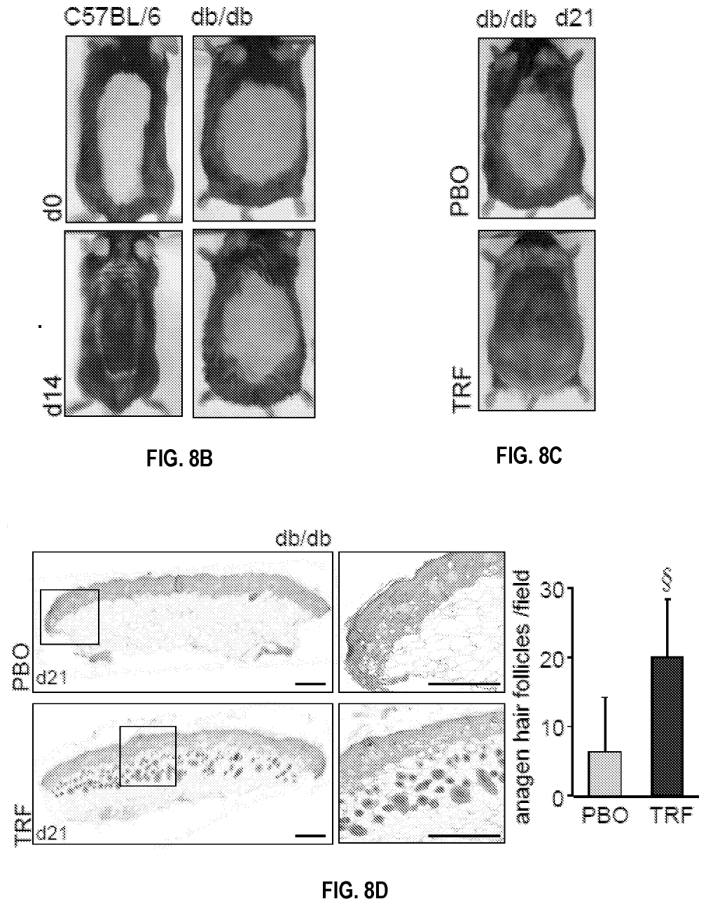

Topical application of tocotrienol rich fraction (TRF) on depilated murine dorsal skin induced anagen hair growth as detected by skin color change from pink to black (FIG. 1A). Higher magnification images using Dermascope™ (Cyberderm) showed thick black hair shaft of anagen hairs in the TRF treated group compared to telogen hairs of the corresponding PBO group at day 21 (FIG. 1B). Hematoxylin and Eosin staining revealed significant increase in the number of anagen hair follicles extending deep to subcutaneous fat following TRF treatment (FIG. 1C, FIG. 8A). Enumeration of hair follicles showed significant increase in number of anagen hair follicles at day 7, 14 and 21 following TRF treatment (FIG. 1C). Anagen transition in leptin receptor-deficient db/db mice is known to be retarded compared to WT mice (FIG. 8B). To study the effect of TRF on anagen hair cycling in db/db mice, TRF was applied on depilated dorsal skin of db/db mice for 21 days. While the PBO mice group remained in telogen phase as noted by the pink color of the dorsal skin, TRF-treated mice showed induction of anagen as evident by the black color of dorsal skin at day 21 (FIG. 8C). Histomorphometric quantitation of the number of anagen hair follicles in db/db mice showed significant increase in TRF group compared with the corresponding PBO group where the hair follicles remained in telogen phase. (FIG. 8D).

Developmental Hair Folliculogenesis in the Adult Skin

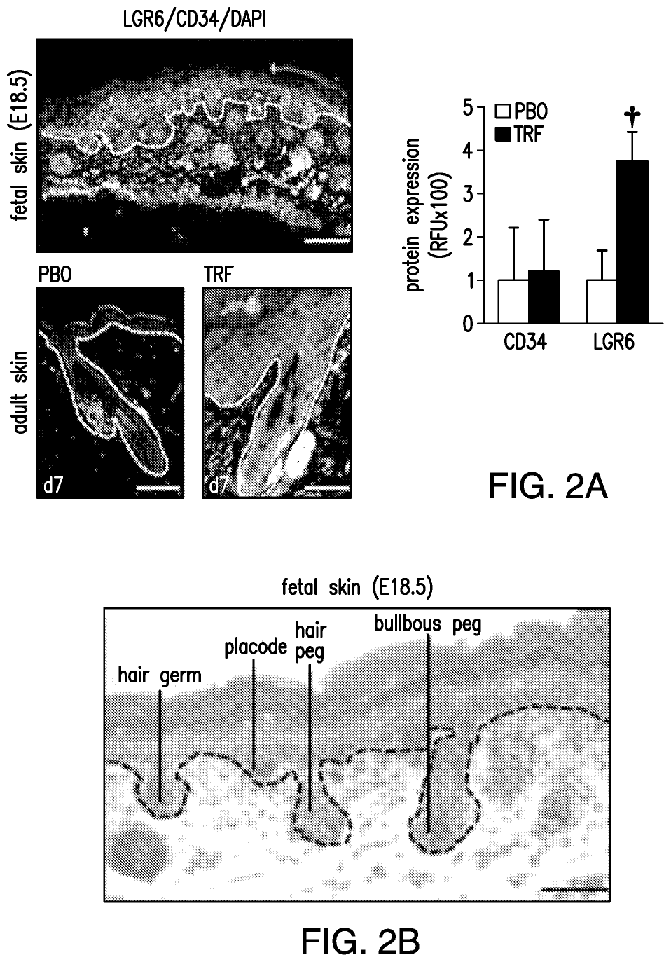

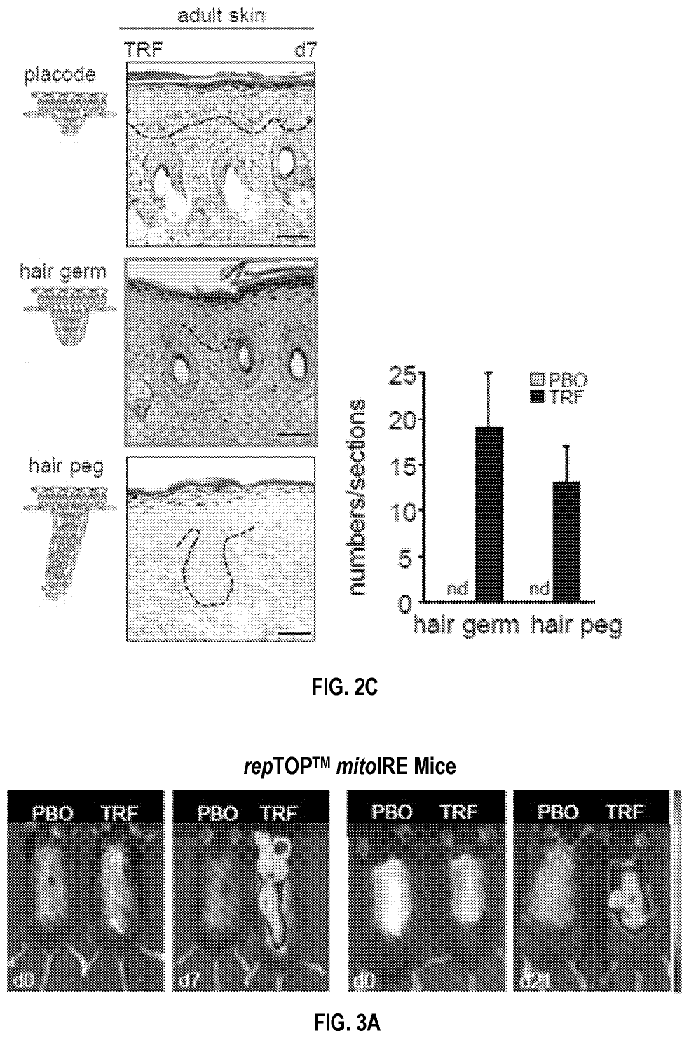

LGR6, a fetal stem cell marker, is abundant in fetal murine skin (FIG. 2A). TRF induced LGR6 in the adult skin (FIG. 2A). Characteristic features of hair follicle development such as “placode”, “hair germ”, “hair peg” and “bulbous peg” were evident in murine fetal skin (E18.5) (FIG. 2B). Such characteristics were not shared by the adult skin. However, topical TRF application for 7 days resulted in the manifestation of above-mentioned fetal characteristics of hair follicular development in the adult skin (FIG. 2C).

Epidermal Keratinocyte Proliferation

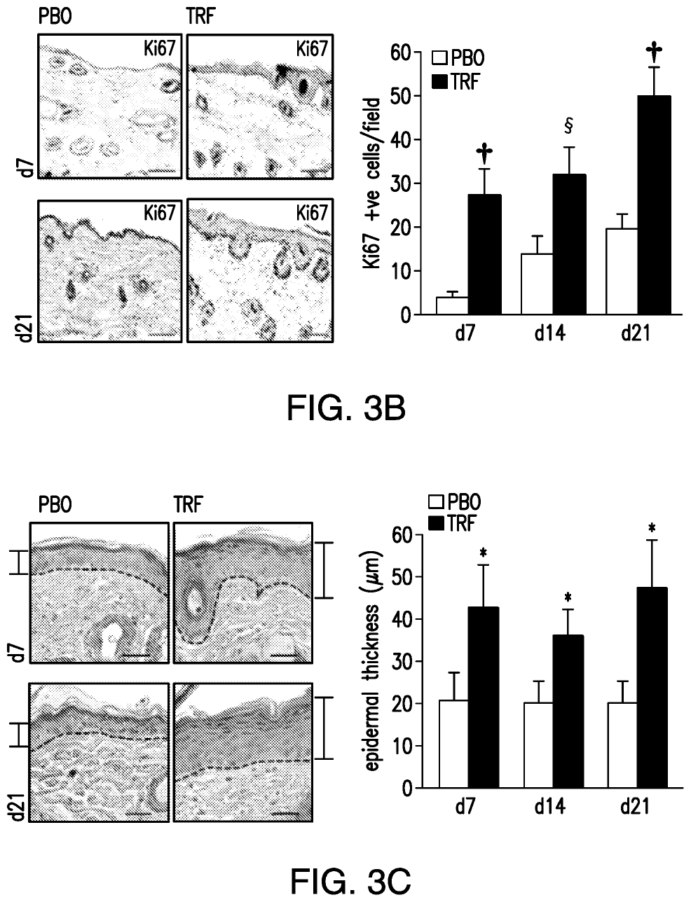

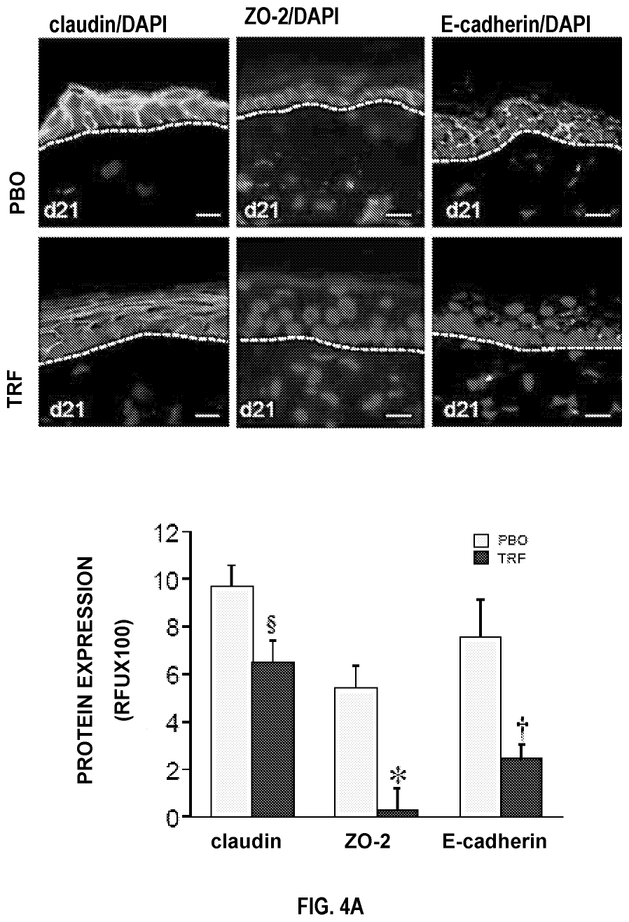

While sharply minimized in homeostatic adult skin, active cell proliferation is a hallmark characteristic of developing fetal skin. Topical application of TRF stimulated skin cell proliferation as detected in repTOP™mitoIRE mice using In Vivo Imaging System (IVIS) (FIG. 3A). This observation was substantiated by immunohistochemical studies demonstrating the presence of Ki67⁺ cells in the epidermis and hair follicles (FIG. 3B). Such keratinocyte proliferation caused by topical TRF application resulted in epidermal thickening (FIG. 3C).

Epidermal Junctional Proteins Expression in Hair Folliculogenesis

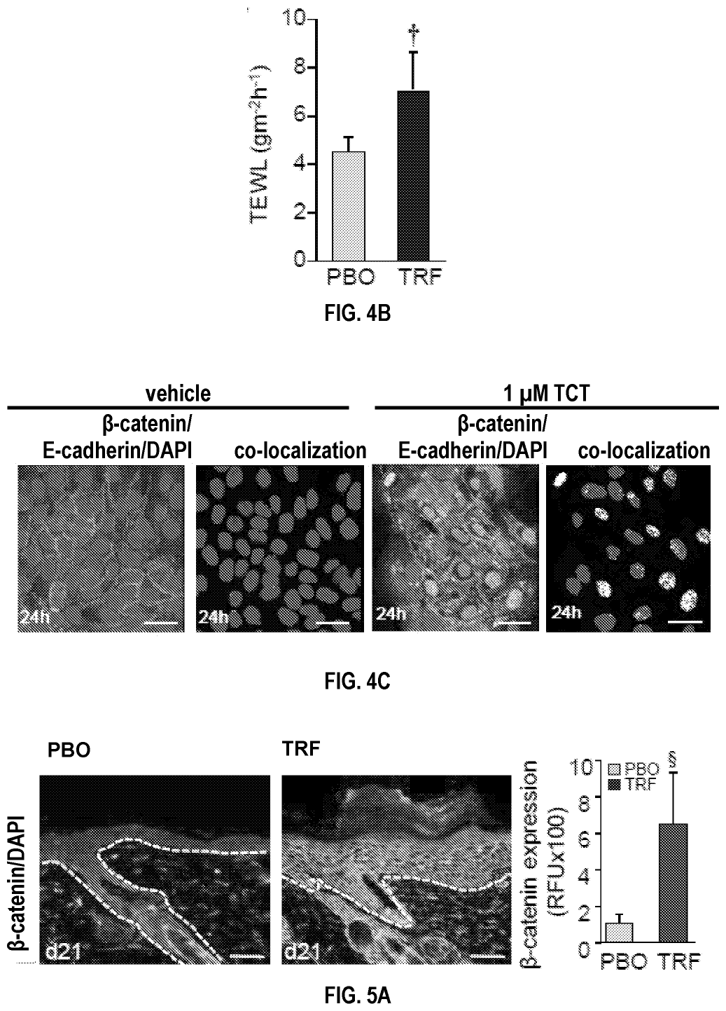

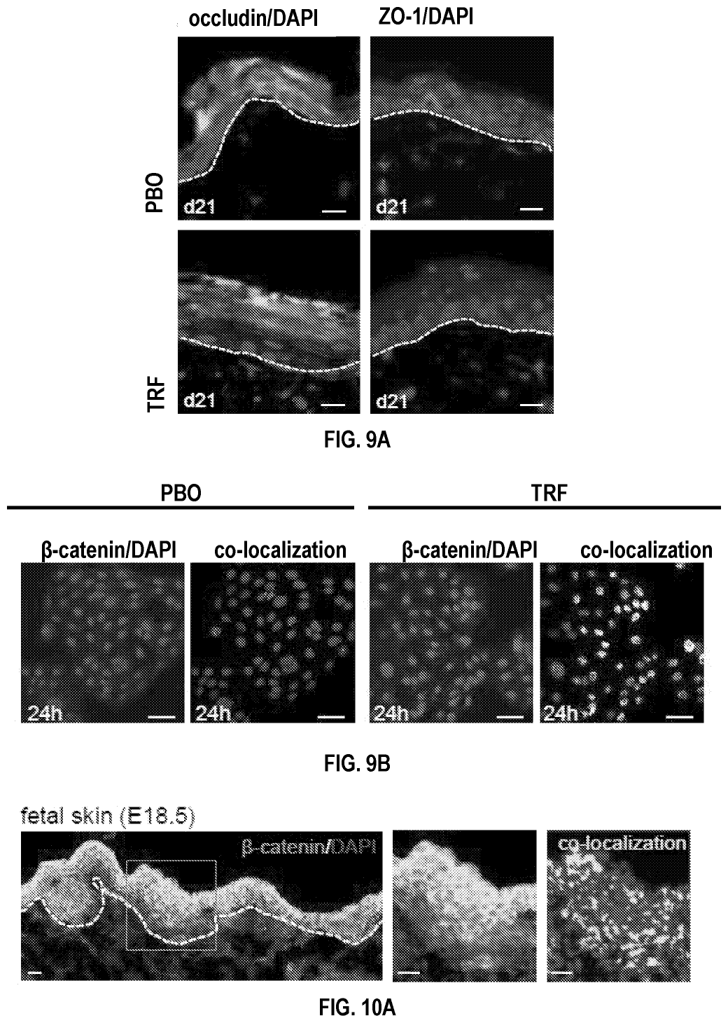

Of the five junctional proteins studied i.e. claudin, occludins, ZO-1, ZO-2 and E-cadherin, topical TRF treatment significantly decreased the expression of claudin, ZO-2 and E-cadherin expression (FIG. 4A). No such change was observed in ZO-1 and occludin (FIG. 9A). Consistent with its effect on suppressing the above mentioned three junctional proteins, topical TRF treatment partly compromised skin barrier function (FIG. 4B). In vitro studies with human HaCaT keratinocytes showed that exposure to pure tocotrienol causes sharp decrease in membrane E-cadherin expression associated with nuclear translocation of β-catenin (FIG. 4C). Such nuclear translocation of β-catenin was also observed when HaCaT cells were exposed to TRF (FIG. 9B).

Inducible β-Catenin Expression and Nuclear Translocation

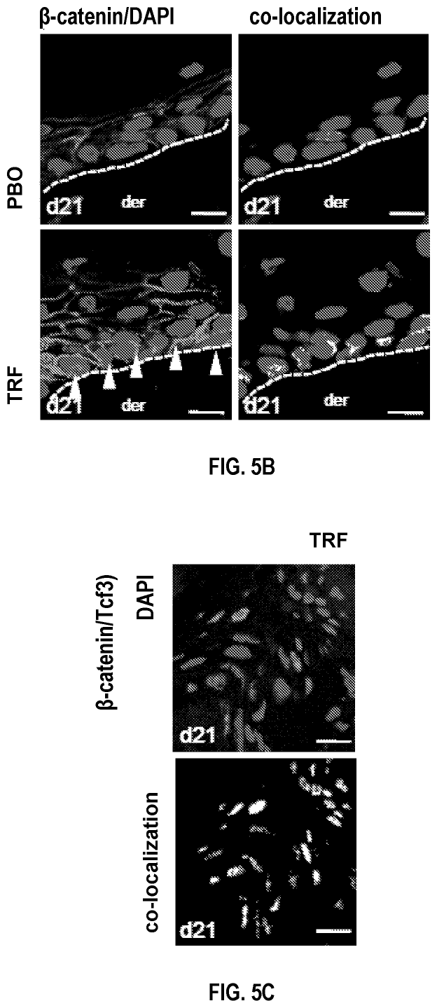

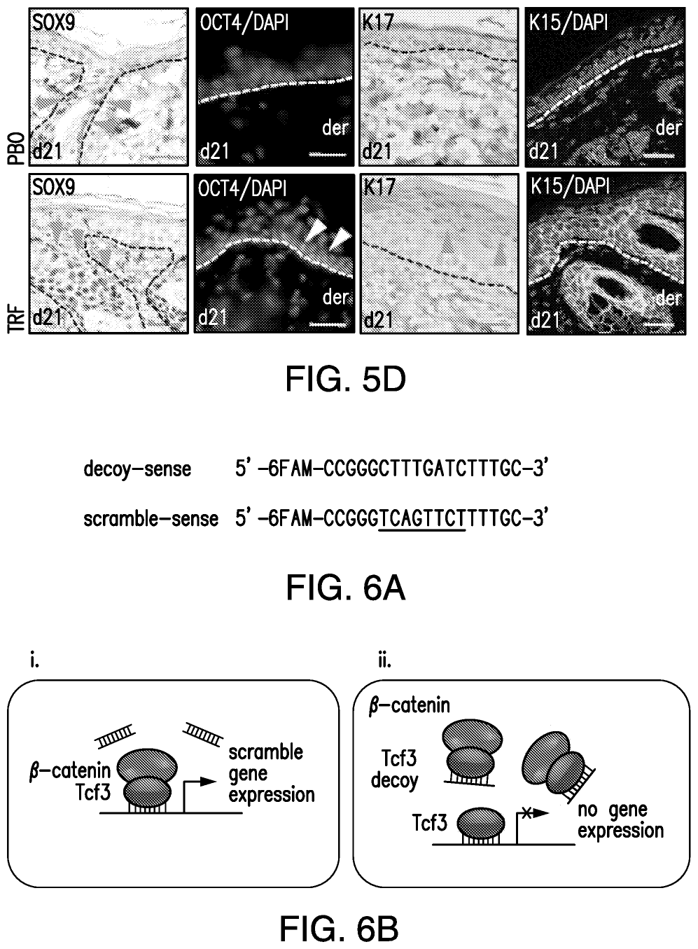

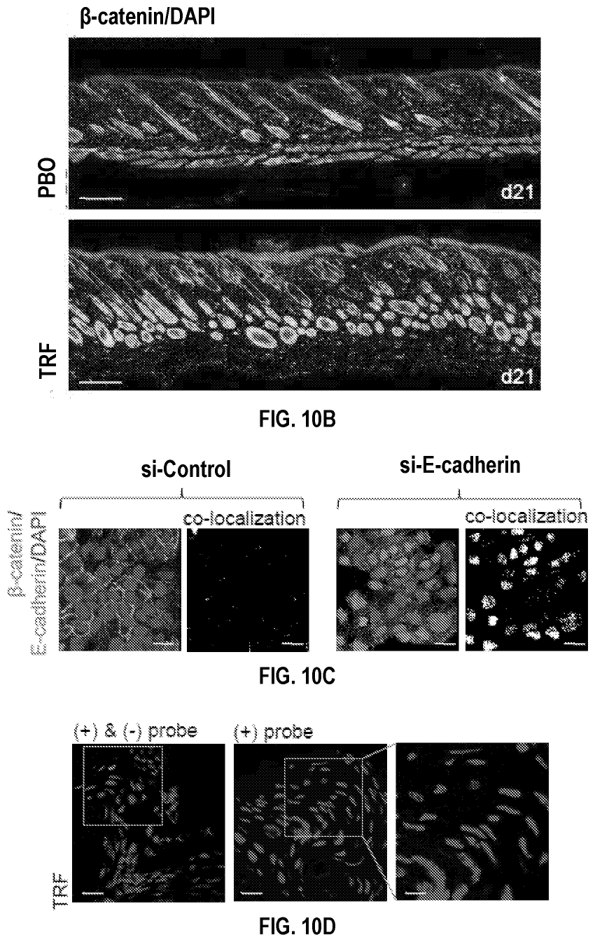

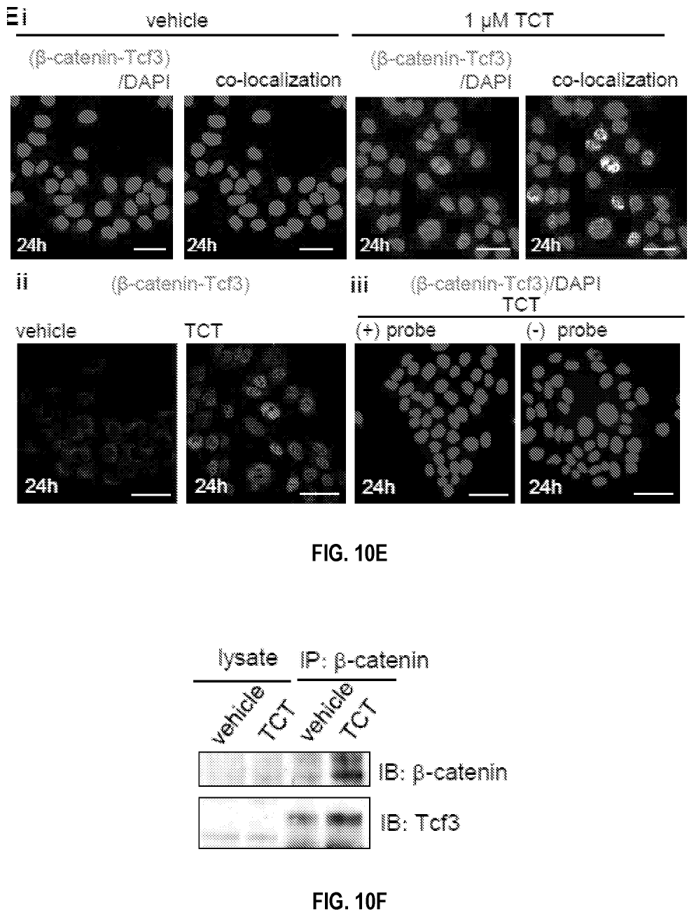

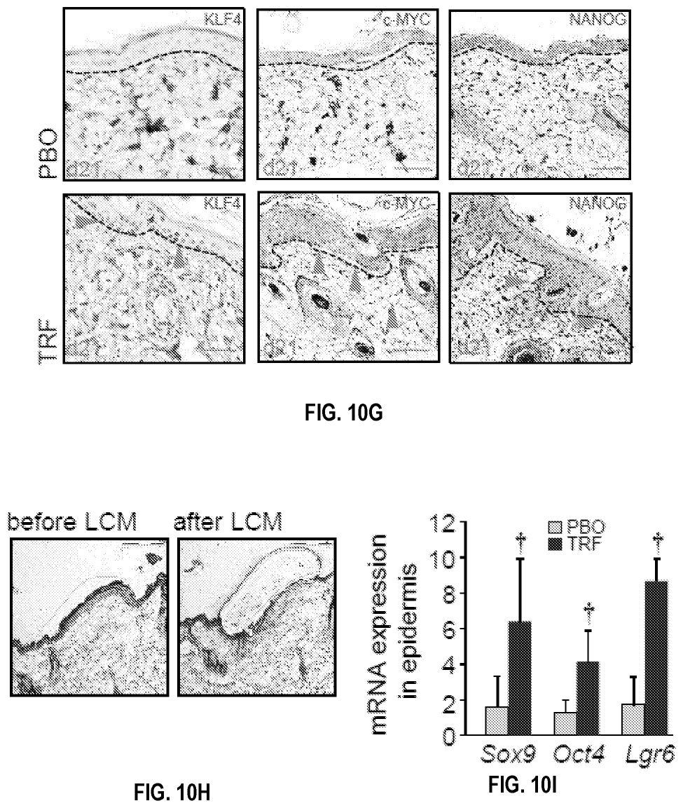

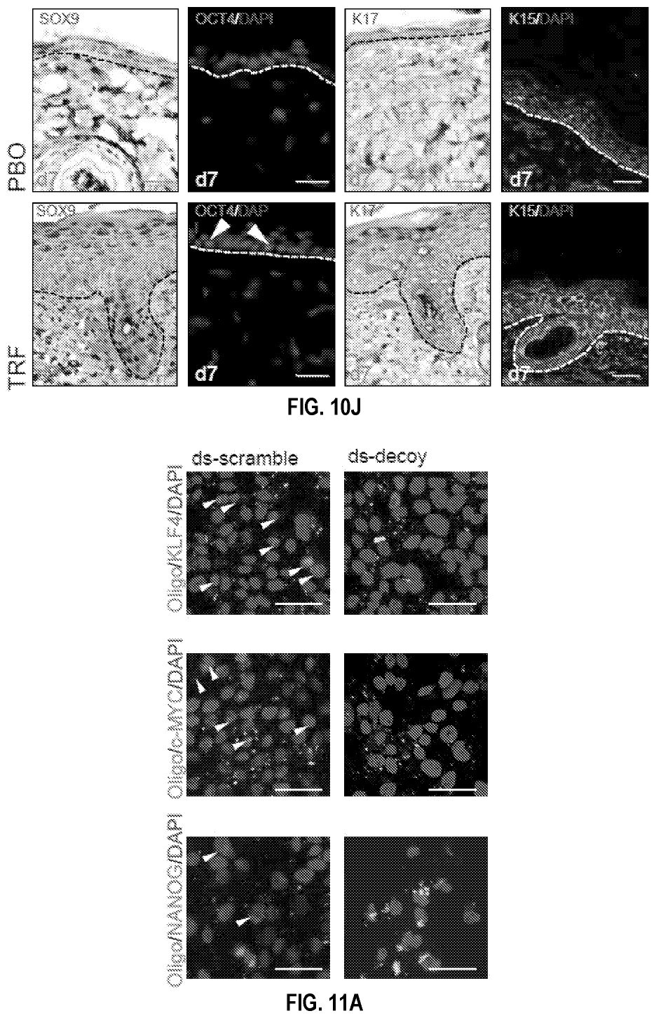

In developmental folliculogenesis, β-catenin represents a major signaling hub. In keratinocytes of developing fetal skin, activated β-catenin is primarily localized in the nucleus (FIG. 10A). Topical application of TRF on adult murine skin resulted in potent induction and activation of epidermal β-catenin as manifested by higher expression and nuclear translocation (FIGS. 5A&B, FIG. 10B). Such observation, akin to the fetal phenotype, is in contrast with what is typically observed in the resting adult skin (FIG. 10B). In human keratinocytes, knock-down of E-cadherin caused translocation of membrane and cytosolic β-catenin to the nucleus (FIG. 10C). In the nucleus, β-catenin directly interacted with the transcription factor 3 (Tcf3) (FIG. 5C, FIGS. 10D-F). Tcf3 is known to repress pluripotency factors. Functional significance of observed β-catenin-Tcf3 binding and sequestration of Tcf3 following TRF treatment was defined by the observation demonstrating elevated levels of skin specific pluripotency factors KLF4, c-MYC and NANOG in epidermal cells (FIG. 10G). Gene expression data from laser captured epidermis (FIG. 10H) revealed that TRF induced binding of β-catenin with Tcf3 resulted in upregulation of sox9, oct4 and lgr6 (FIG. 10I). Furthermore, immunohistochemical analyses showed robust expression of SOX9, OCT4, K17, and K15 in the TRF-treated adult murine epidermis (FIG. 5D and FIG. 10J).

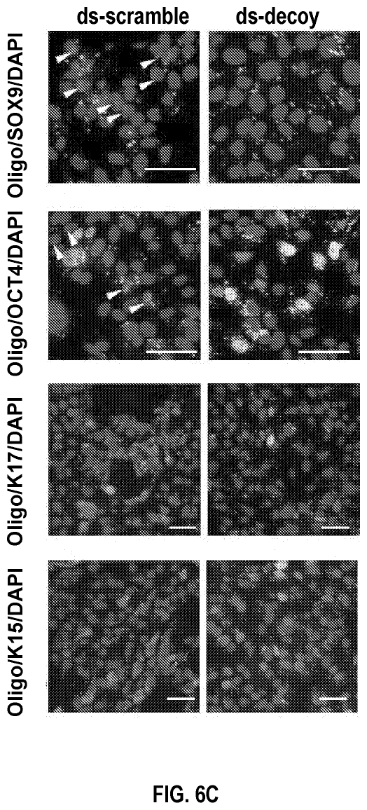

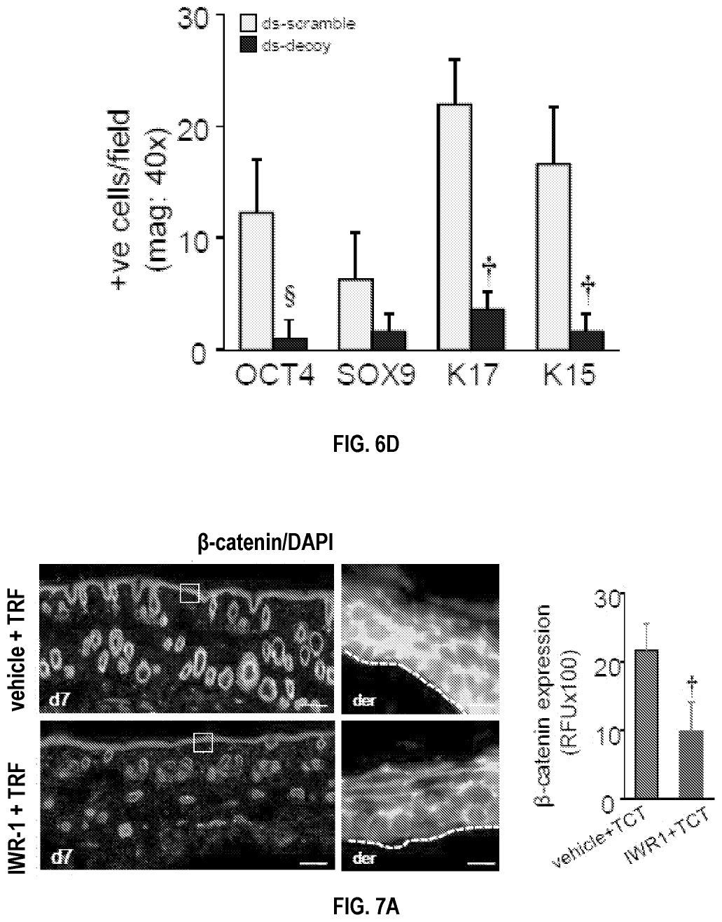

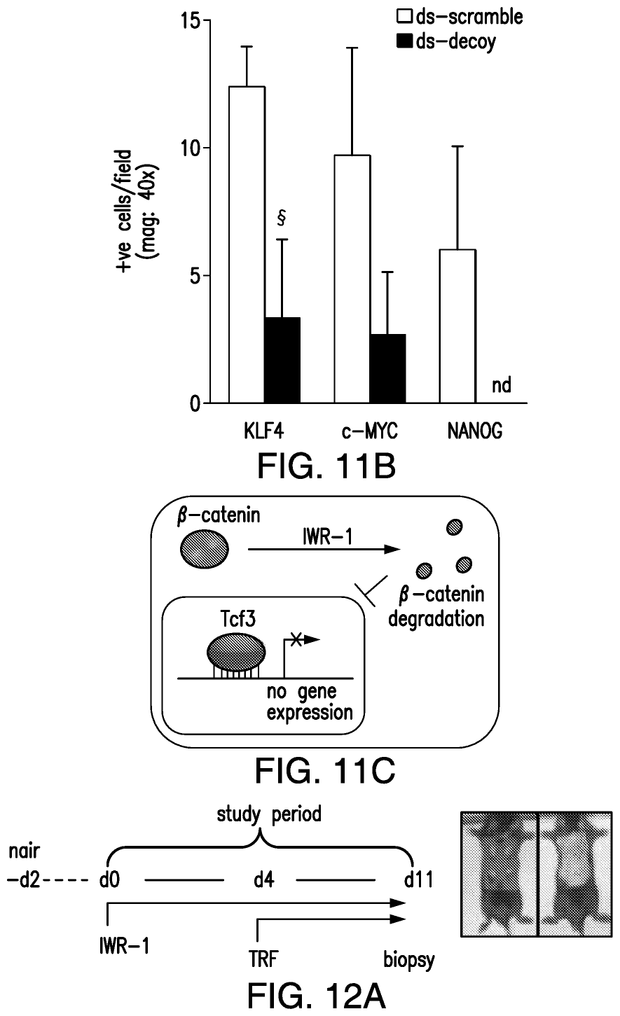

Significance of β-catenin-Tcf3 interaction in induction of pluripotency. A FAM labelled oligodeoxynucleotide construct, based on Tcf3 binding consensus sequence as described previously, was used as decoy to circumvent transactivation caused by the β-catenin-Tcf3 complex. Negative-control oligodeoxynucleotides with eight-base mismatch served as scrambled control (FIG. 6A). This strategy was based on the competition of β-catenin-bound Tcf3, under conditions of tocotrienol treatment, to bind to endogenous target sequence in the presence of exogenous abundant Tcf3 decoy (FIG. 6B). In the presence of the above-mentioned decoy double-stranded oligonucleotide, the induction of stemness as well as pluripotency factors in response to tocotrienol treatment was markedly blunted. Quantitative immunocytochemistry results for SOX9, OCT4, K17 and K15 are shown in FIG. 6C. Additionally, findings on the expression of KLF4, c-MYC and NANOG in human keratinocytes are presented (FIGS. 11A&B).

Pharmacological Inhibition of Inducible Anagen Hair Cycling

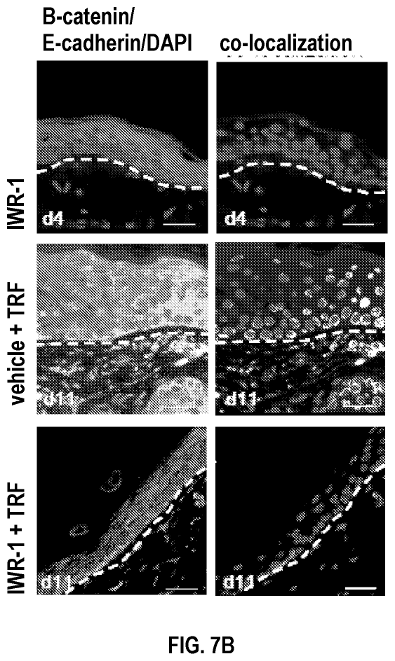

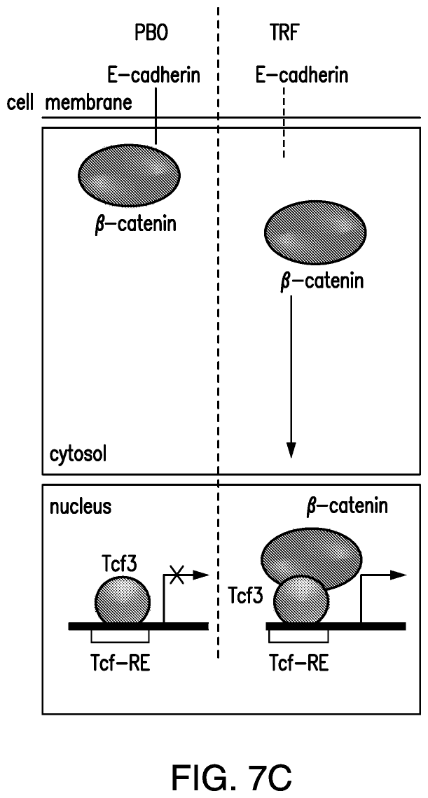

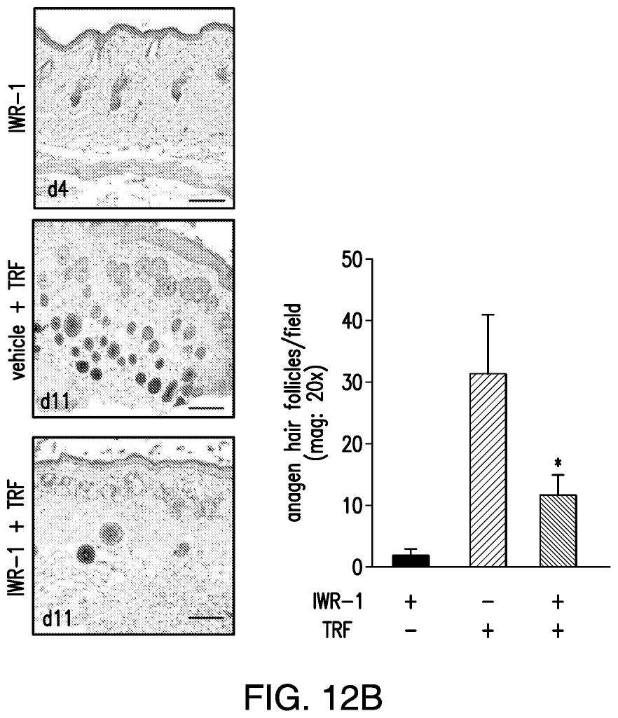

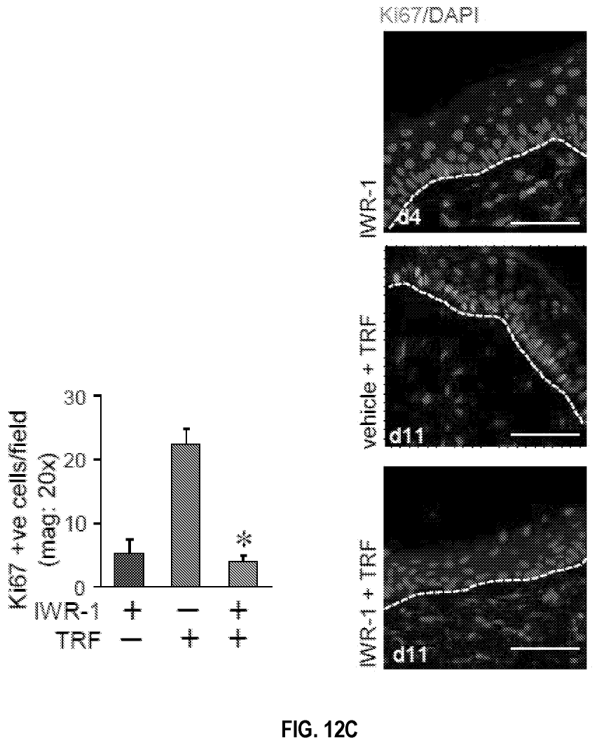

The significance of β-catenin in TRF induced anagen hair cycling in adult murine skin was tested using IWR-1, a pharmacological inhibitor of β-catenin (FIG. 11C). Topical pre-treatment of the murine adult skin with IWR-1 (FIG. 12A) significantly decreased the number of anagen hair follicles induced by topical TRF application (FIG. 12B). Such observation was associated with the finding that IWR-1 is also capable of eliminating the effect of TRF on stimulating keratinocytes proliferation (FIG. 12C). Topical IWR-1 pre-treatment was effective in degrading epidermal β-catenin (FIG. 7A). Interestingly, IWR-1 pre-treatment did not influence the lowering of E-cadherin in response to TRF (FIG. 7B). Thus, the ability of topical TRF treatment to induce epidermal proliferation and hair folliculogenesis in the adult skin was significantly impaired by IWR-1 (FIGS. 12B&C). This observation suggests that in the pathway of TRF induced folliculogenesis, E-cadherin resides upstream of β-catenin (FIG. 7C).

### Discussion

The hair follicles serve multiple critical functions. They undergo cyclic transformations between phases of rapid growth (anagen), apoptosis-driven regression (catagen) and relative quiescence (telogen). With every cycle, the hair follicle regenerates itself. The result is daughter-cell populations which are derived from resident epithelial, neural, and mesenchymal stem cells. In adults, during each cycle, a new hair shaft is formed, and the old hair is eventually shed mostly by an actively regulated exogen. In the adult skin, generation of the new hair shaft depends on the activation of hair-specific epithelial stem cells that are harbored in the bulge region of the hair follicle epithelium. Development of hair follicles during fetal skin morphogenesis is preceded by active epidermal cell proliferation. Although the process of adult hair follicle and fetal hair follicle formation have several contrasting features, it is evident that the adult skin may acquire plasticity by activation of key signaling molecules such as β-catenin.

β-Catenin has emerged as a key signaling hub for hair follicles formation in both adult as well as in fetal states. Transient β-catenin activation advances telogen to anagen. Within epidermal keratinocytes, β-catenin is localized in three major compartments, the plasma membrane, cytoplasm and in the nucleus. The β-catenin signaling pathway is evolutionarily conserved. Nuclear translocation followed by subsequent binding to repressor transcription factor Tcf3 assigns the function of transcriptional activators to β-catenin. Epithelial integrity during adult skin homeostasis is achieved by the binding of β-catenin with E-cadherin in the plasma membrane. Such membrane binding with the intracellular domain of E-cadherin restrains the function of β-catenin as transcriptional activator. Thus E-cadherin plays critical role in the stabilization and in determining the function of β-catenin. This example reports the first evidence demonstrating that conditions resulting in the loss of membrane E-cadherin may release and activate β-catenin. Interestingly, such form of β-catenin activation was achieved by the topical application of a phytochemical. Pure tocotrienol, the seed form of natural vitamin E which is a major component of TRF, markedly depleted membrane E-cadherin releasing β-catenin for nuclear translocation. Under such conditions, nuclear β-catenin binds to Tcf3 blocking its repressive action on cell plasticity factors. As a result, cell stemness and pluripotency was induced. Such effect of tocotrienol was blunted in the presence of a decoy oligonucleotide designed to limit the binding of Tcf3 to its endogenous consensus sites establishing the withdrawal of Tcf3 as transcription factor as a central mechanism responsible for tocotrienol action.

In vivo studies showed that TRF application decreased junctional protein E-cadherin in the skin. Released from the membrane, β-catenin becomes available for nuclear translocation. This process can be intercepted by degradation of cytosolic β-catenin by the pharmacological inhibitor IWR-1. Thus, TRF-induced mobilization of β-catenin did not influence the Tcf3 function in the presence of IWR-1. This observation demonstrates that the effect of TRF on hair follicle formation is dependent on loss of E-cadherin and activation of β-catenin. The study of TRF function helped elucidate a novel β-catenin pathway that relies on the loss of upstream E-cadherin for its activation. Epithelial placodes, characteristic of the murine embryonic hair follicle development, invaginate to give rise to the germ by E15.5, the peg by E17.5, and the bulbous peg by E18.5. This work reports first evidence of placode, hair peg and hair germ during the course of adult hair folliculogenesis. Once the bud proliferates, it encases the dermal papilla, and further differentiates to form the hair shaft.

Diabetic complications include impaired induction of anagen. Leptin, an adiponectin, also acts as anagen inducer. Leptin receptor is highly expressed in dermal cells including the dermal papilla that is critical for hair follicle morphogenesis. During hair follicle development, dermal papilla are formed by epithelial condensate to form mature dermal papilla. This example shows that it is possible to induce anagen under diabetic conditions. This observation supports the contention that TRF, the inducer of anagen in the diabetic skin, acts upstream of dermal papilla formation to induce anagen. Given that dermal papilla formation is necessary for the induction of anagen in the adult skin, this observation leads to the notion that TRF induces anagen in the diabetic skin via a pathway akin to that during fetal skin development. Such pathway involves activation of β-catenin and the formation of placode.

The post-natal skin does not generate any new hair follicles. Thus, growth of hair follicles in the adult skin relies on the hair follicular regeneration process contributed by the stem cell niche of existing follicles. To serve this purpose, and for the maintenance of epidermis, reservoirs of multipotent epithelial stem cells are set aside at the base of the hair follicle ‘bulge’. Follicle stem cells are activated at the telogen-to-anagen transition, to initiate a new round of hair growth. Hair follicular stem cells undergo multiple sessions of activity during which hair and skin regeneration occurs. Hair follicles are a major contributor to the overall stem cell pool of the adult skin. Thus, follicular growth in the adult skin may markedly enhance stem cell abundance in this largest organ of the body. A rapidly growing body evidence directly implicate follicular stem cells in skin function such as turnover and maintenance of the adult skin, management of age-related complications, improved tissue repair and protection against UV damage. It is therefore plausible that substantial induction of hair follicles in the adult skin by TRF will influence skin function and fate as it relates to the above-mentioned conditions, complications and their derivatives such as sunburn, melanoma, and psoriasis.

Both murine and human hair follicles have comparable cell type that undergoes repetitive cycle of active growth (anagen), regression (catagen), and quiescence (telogen). However, one of the major differences between human and murine hair follicle is that in the mouse skin the anagen phase lasts for only 2-3 weeks whereas in humans it last for several years. Consistent with the finding of this work, Beoy et al. reported induction of hair growth by TRF in humans. TRF represents a rich natural source of tocotrienol that is readily accessible and generally recognized as safe by the FDA (GRN No. 307). It is thus well suited for human intervention. Therefore, all in vivo studies in this work were conducted using TRF. Emphasis on elucidating the mechanistic underpinnings of how TRF induced anagen hair cycling necessitated the study of pure α-tocotrienol, a major component of TRF, in all in vitro experiments.

In summary, this example presents the first evidence on a number of fronts demonstrating that it is possible to induce anagen hair follicle development in the adult skin via a pathway akin to that during fetal skin development. Such pathway, inducible by topical phytochemical treatment, is capable of inducing follicular growth in the adult diabetic skin which is otherwise known to be refractory to induction of anagen. Downregulation of epithelial E-cadherin is recognized as a trigger for β-catenin activation which sequesters Tcf3 unleashing hair follicular regeneration. Above and beyond its role on growth of hair, hair follicles serve as stem cell reservoirs that may influence numerous aspects of skin function and complication. Thus, novel strategies to induce follicular proliferation in the adult skin are likely to have a wide range of implications in preserving and restoring skin function.

### Materials and Methods

Animal and experimental design. Male C57BL/6 mice were obtained from Harlan Laboratory (Indianapolis, Ind.). Male mice homozygous (BKS.Cg-m^{+/+} Lepr^{db/J,} or db/db; stock no 000642) for spontaneous mutation of the leptin receptor (Lepr^{db}) were obtained from Jackson Laboratory. Male repTOP™mitoIRE mice were obtained from Charles River laboratory. These mice have a luciferase reporter tagged with the cyclin B2 promoter. Thus administration of luciferin at a dose of 100 mg/kg body weight i.p (intraperitoneally) produces bioluminescence from any proliferating cell of the body.⁵³ All animals were 7-8 weeks old at the start of the experiment. All animal studies were performed in accordance with protocols approved by the Laboratory Animal Care and Use Committee of The Ohio State University.

Tocotrienol rich fraction (TRF) was obtained from commercially available Tocovid Suprabio capsules.⁵⁴ Each capsule contains Tocomin 50% which typically provides d-Alpha-Tocotrienol 61.52 mg, d-Gamma-Tocotrienol 112.80 mg, d-Delta-Tocotrienol 25.68 mg, d-Alpha-Tocopherol 91.60 IU, Plant Squalene 51.28 mg, Phytosterol Complex 20.48 mg, Phytocarotenoid Complex 360.00 ug. Thus, each capsule contains 144.86 mM of d-Alpha-Tocotrienol. In the placebo, the tocotrienol was replaced by soya oil. For in vitro experiments, pure tocotrienol was provided by Excelvite Inc, New Jersey.

Mice were randomly (www.random.org) divided into different groups as indicated in corresponding figure legends. The dorsal skin was shaved and hair was depilated using Nair® two days before experiments. TRF or its placebo (PBO) was applied topically at a dose of 5 mg/cm² skin, thrice per week. During the procedure, mice were anesthetized by low-dose isoflurane inhalation. Digital photographs were collected (Canon PowerShot S6 and Dermascope from Dino-lite ProII). In some experiments, to inhibit β-catenin expression, IWR-1 (Sigma; I0161) was used. A 10% DMSO stock IWR-1 solution was prepared and diluted with glycerol to a final concentration of 2 μg per 0.5 cm² of skin. This solution was applied topically on the dorsal skin of C57B1/6 mice at a dose of 12.5 μL/0.5 cm² for four consecutive days of IWR-1 or vehicle followed by 7 consecutive days of IWR-1 or vehicle for one hour under occlusive dressing (Tegaderm™, 3M, St. Paul, Minn.). Such inhibitor treatment was followed by topical TRF or PBO application as indicated in figure legends.

In vivo imaging. Fifteen minutes before imaging, the repTOP™mitoIRE mice were injected intraperitoneally with the Potassium salt of Beetle luciferin.⁵³ The animals were imaged under anesthesia using IVIS Lumina II optical imaging system and the overlay images were made using Living Image software.

Detection of anagen induction. Promotion of hair growth was evaluated by observing the skin color which is indicative of the telogen-to-anagen conversion.⁵⁰ Skin, collected and 8 μm formalin-fixed paraffin-embedded skin sections, were deparaffinized and stained with hematoxylin & eosin. Mosaic images at 20× were collected using Axioscanner (Zeiss Microscopy). From histological characterization of the sections, anagen follicles in dermis and subcutis layers were enumerated.

Cell and cell culture. Immortalized human keratinocytes (HaCaT) were grown in Dulbecco's low-glucose modified Eagle's medium (Life Technologies) as described previously.⁵⁵ Cells were maintained in a standard culture incubator with humidified air containing 5% CO₂ at 37° C.

Synthesis of Tcf3 decoy and control double-stranded oligodeoxynucleotides. Tcf3 decoy and control double-stranded oligodeoxynucleotides were prepared as described previously.²² Briefly, FAM labelled phosphorothioate oligodeoxynucleotides were synthesized and purified by Sigma-Aldrich. The oligonucleotides sequence used were as follow: Tcf3 decoy sense, 5′-6FAM-CCGGGCTTTGATCTTTGC-3′ (SEQ ID NO:1); Tcf3 decoy anti-sense, 5′-6FAM-GCAAAGATCAAAGCCCGG-3′ (SEQ ID NO:2); Tcf3 scramble sense, 5′-6FAM-CCGGGTCAGTTCTTTTGC -3′ (SEQ ID NO:3); Tcf3 scramble anti-sense, 5′-6FAM-GCAAAAGAACTGACCCGG -3′ (SEQ ID NO:4). Double-stranded oligodeoxynucleotides were prepared by dissolving sense and antisense oligodeoxynucleotides were dissolved in TE buffer (IDT) at a concentration of 1 mmol/L. Each sense-antisense pair was annealed by heating at 95° C. for 10 minutes. The reaction mixture was then allowed to cool to room temperature.

Transfection of siRNA and double-stranded oligodeoxynucleotides. DharmaFECT™ 1 transfection reagent was employed to transfect HaCaT cells with on target siRNA for E-cadherin (Dharmacon) as described previously.⁵⁶ Cells were re-seeded on to chamber slides 48 h after transfection. Next, cells were fixed after 24 h or 72 h after transfection. Double-stranded oligodeoxynucleotides were transfected to HaCaT cells using Lipofectamine™ LTX/plus reagent (Invitrogen) as described previously.¹¹ Cells were processed for immunocytochemistry 48 h after transfection.¹¹

Trans-epidermal water loss (TEWL). DermaLab TEWL Probe (cyberDERM inc., Broomall) was used to measure the trans-epidermal water loss from the skin as described previously.⁵⁷ TEWL was expressed as (gm⁻²h⁻¹).

In situ proximity ligation assay (PLA). PLA was performed as described previously using Sigma Duolink® in situ red starter kit for goat/rabbit (DUO92105) following manufacturer's instructions except that the incubation time was prolonged to 90 minutes.⁵⁸ The assay was performed using rabbit Tcf3 (Abcam, ab69999; 1:50) and mouse β-catenin (Abcam, ab22656; 1:200) antibodies. Slides were imaged using Olympus FV 1000 spectral confocal microscope.

Laser capture microdissection of the epidermis. Laser capture microdissection was performed using a laser microdissection system from PALM Technologies (Bernreid, Germany) containing a PALM MicroBeam and RoboStage for high-throughput sample collection and a PALM RoboMover (PALM Robo software, Version 2.2) as described previously.⁵⁹ For epidermal LCM, sections were stained with hematoxylin for 30 s, subsequently washed with DEPC-H₂O and dehydrated in ethanol. The epidermis was identified based on the histology. Epidermal tissue elements were typically cut and captured under a 20× ocular lens. Samples were catapulted into 25 μl of cell direct lysis extraction buffer (Invitrogen). Approximately 100,000 μm² of tissue area was captured into each cap and the extract was then held at −80° C. for further analyses.

Quantitative real-time PCR. For mRNA expression studies, total cDNA synthesis was achieved using the SuperScript®Vilo™ cDNA Synthesis kit (Invitrogen). The transcript levels of Oct4, Sox9, and LGR6 were assessed by real time PCR using SYBR Green-I (Applied Biosystems). GAPDH served as housekeeping controls. The following primer sets were used: m_GAPDH F, 5′-ATGACCACAGTCCATGCCATCACT -3′ (SEQ ID NO:5); m_GAPDH R, 5′-TGTTGAAGTCGCAGGAGACAACCT -3′ (SEQ ID NO:6); m_Oct4 F, 5′-TGGATCCTCGAACCTGGCTA -3′ (SEQ ID NO:7); m_Oct4 R, 5′-CTCAGGCT GCAAAGTCTCCA-3′ (SEQ ID NO:8); m_Sox9 F, 5′-CCCCATCGACTTCCGCGACG-3′ (SEQ ID NO:9); m_Sox9 R, 5′-TGGGTGCGGTGCTGCTGATG-3′ (SEQ ID NO:10); m_LGR6 F, 5′-CGTCGGTGCTGCTGCTCACA -3′ (SEQ ID NO:11); m_LGR6 R, 5-CGGCCACCACCAGGAAGCAG -3′ (SEQ ID NO:12).

Immunoprecipitation and Immunoblots. HaCaT cells (0.5×10⁶ cells/well) were seeded in six-well plates and treated with either 1 μM TCT or equivalent volume of ethanol for 24 h. Immunoprecipitation and subsequent immunoblots were performed as described previously.¹¹ Briefly, the nuclear fractions from the cells were extracted (Signosis Nuclear Extraction Kit, SK001) and 100 μg of pooled nuclear extract were incubated with 500 ng β-catenin antibody (abcam; ab32571) for overnight at 4° C. and then incubated at 4° C. with 30 μl of anti-rabbit IgG beads (TrueBlot Ig IP beads; eBioscience). Immunoprecipitated complexes were washed four times with lysis buffer (centrifugation at 1000×g at 4° C. for 5 min), recovered in 25 μl of 4×Laemmli buffer with 50 mM of fresh dithiothreitol and boiled for 10 min. Next, equal volume of samples was loaded onto SDS-PAGE gel and immunoblot.

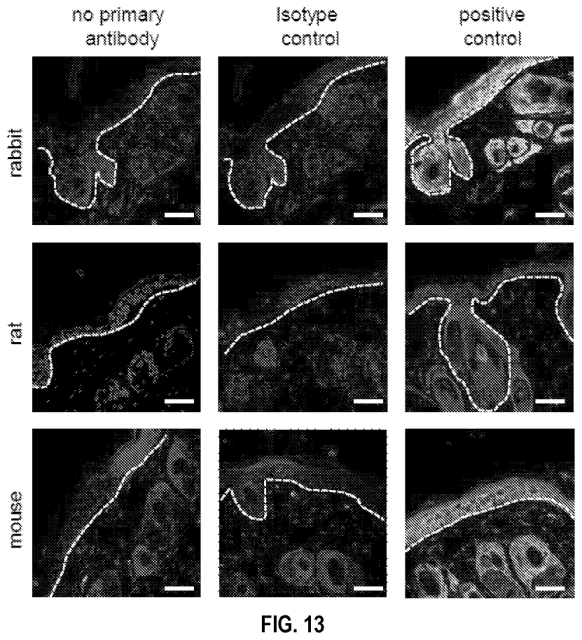

Histology, immunohistochemistry (IHC) and immunocytochemistry (ICC). Histology of skin was performed from 8 μm thick paraffin sections after staining with Hematoxylin and Eosin (H&E). Immunostaining of Ki67 (Abcam, ab15580; 1:400), LGR6 (Abcam, ab126747; 1:100), CD34 (Abcam, ab8158; 1:200), OCT4 (Abcam, ab19857; 1:200), SOX9 (Abcam, ab185230; 1:1000), KLF4 (Abcam; ab151733, 1:200), c-MYC (Abcam; ab32072, 1:200), NANOG (Abcam; ab80892, 1:200), K17 (Abcam, ab53707; 1:100), K15 (Abcam, ab52816; 1:100), Claudin (Invitrogen, RB9209P; 1:200), Occludin (Invitrogen, 711500; 1:200), ZO-1(Invitrogen, 617300; 1:200), ZO-2 (Invitrogen, 389100; 1:200), E-cadherin (Life Technologies, 131900; 1:200) and β-catenin (Abcam, ab22656; 1:200) were performed on paraffin and cryosections of skin sample using specific antibodies as indicated.⁵⁷ For immunocytochemistry, cells were fixed with cell fixation buffer (ebiosciences; 00-8222-49) and stained for respective antibodies. Specificity of the antibodies were validated using rabbit isotype control (Abcam, ab27478; 1:400), rat isotype control (Abcam, ab18412; 1:200), and mouse isotype control (Abcam, ab18443; 1:200) (FIG. 13). Briefly, OCT embedded tissue were cryosectioned (10 μm), fixed with cold acetone, blocked with 10% normal goat serum and incubated with specific antibodies overnight at 4° C. Signal was visualized by subsequent incubation with either biotinylated-tagged for DAB staining and fluorescence-tagged appropriate secondary antibodies (Alexa 488-tagged α-mouse, 1:200; Alexa 568-tagged α-mouse, 1:200; Alexa 488-tagged α-rabbit, 1:200; Alexa 568-tagged α-rabbit, 1:200; Alexa 568-tagged α-rat, 1:200).

Statistical analyses. Data are expressed as mean±SD of at least four to six animals per group as indicated in figure legends. Significance between two groups was tested using Student's t-test (two-tailed). A value p<0.05 was considered statistically significant. Significant responses (p<0.05) are marked by symbols (†, §, *) and their corresponding p values are provided in figure legends.

### REFERENCES

1. Mistriotis, P, and Andreadis, S T (2013). Hair Follicle: A Novel Source of Multipotent Stem Cells for Tissue Engineering and Regenerative Medicine. Tissue Engineering Part B, Reviews 19: 265-278.

2. Schneider, M R, Schmidt-Ullrich, R, and Paus, R (2009). The Hair Follicle as a Dynamic Miniorgan. Current Biology 19: R132-R142.

3. Millar, S E (2002). Molecular mechanisms regulating hair follicle development. The Journal of investigative dermatology 118: 216-225.

4. Fuchs, E (2008). Skin stem cells: rising to the surface. The Journal of cell biology 180: 273-284.

5. McGowan, K M, and Coulombe, P A (1998). Onset of keratin 17 expression coincides with the definition of major epithelial lineages during skin development. The Journal of cell biology 143: 469-486.

6. Snippert, H J, Haegebarth, A, Kasper, M, Jaks, V, van Es, J H, Barker, N, et al. (2010). Lgr6 marks stem cells in the hair follicle that generate all cell lineages of the skin. Science (New York, N.Y.) 327: 1385-1389.

7. Lo Celso, C, Prowse, D M, and Watt, F M (2004). Transient activation of beta-catenin signalling in adult mouse epidermis is sufficient to induce new hair follicles but continuous activation is required to maintain hair follicle tumours. Development (Cambridge, England) 131: 1787-1799.

8. Silva-Vargas, V, Lo Celso, C, Giangreco, A, Ofstad, T, Prowse, D M, Braun, K M, et al. (2005). Beta-catenin and Hedgehog signal strength can specify number and location of hair follicles in adult epidermis without recruitment of bulge stem cells. Developmental cell 9: 121-131.

9. Ito, M, Yang, Z, Andl, T, Cui, C, Kim, N, Millar, S E, et al. (2007). Wnt-dependent de novo hair follicle regeneration in adult mouse skin after wounding. Nature 447: 316-320.

10. Sen, C K, Khanna, S, and Roy, S (2006). Tocotrienols: Vitamin E beyond tocopherols. Life sciences 78: 2088-2098.

11. Khanna, S, Roy, S, Park, H A, and Sen, C K (2007). Regulation of c-Src activity in glutamate-induced neurodegeneration. The Journal of biological chemistry 282: 23482-23490.

12. Khanna, S, Roy, S, Ryu, H, Bahadduri, P, Swaan, P W, Ratan, R R, et al. (2003). Molecular basis of vitamin E action: tocotrienol modulates 12-lipoxygenase, a key mediator of glutamate-induced neurodegeneration. The Journal of biological chemistry 278: 43508-43515.

13. Khanna, S, Roy, S, Slivka, A, Craft, T K, Chaki, S, Rink, C, et al. (2005). Neuroprotective properties of the natural vitamin E alpha-tocotrienol. Stroke; a journal of cerebral circulation 36: 2258-2264.

14. Sen, C K, Khanna, S, and Roy, S (2004). Tocotrienol: the natural vitamin E to defend the nervous system? Annals of the New York Academy of Sciences 1031: 127-142.

15. Sen, C K, Rink, C, and Khanna, S (2010). Palm Oil-Derived Natural Vitamin E α-Tocotrienol in Brain Health and Disease. Journal of the American College of Nutrition 29: 314S-323 S.

16. Beoy, L A, Woei, W J, and Hay, Y K (2010). Effects of Tocotrienol Supplementation on Hair Growth in Human Volunteers. Tropical Life Sciences Research 21: 91-99.

17. Tartaglia, L A, Dembski, M, Weng, X, Deng, N, Culpepper, J, Devos, R, et al. (1995). Identification and expression cloning of a leptin receptor, OB-R. Cell 83: 1263-1271.

18. Li, H-H, Fu, X-B, Zhang, L, and Zhou, G (2008). Comparison of proliferating cells between human adult and fetal eccrine sweat glands. Archives of Dermatological Research 300: 173-176.

19. Tsai, S-Y, Sennett, R, Rezza, A, Clavel, C, Grisanti, L, Zemla, R, et al. (2014). Wnt/β-catenin signaling in dermal condensates is required for hair follicle formation. Developmental biology 385: 179-188.

20. Cole, M F, Johnstone, S E, Newman, J J, Kagey, M H, and Young, R A (2008). Tcf3 is an integral component of the core regulatory circuitry of embryonic stem cells. Genes & development 22: 746-755.

21. Chen, L, and Daley, G Q (2008). Molecular basis of pluripotency. Human molecular genetics 17: R23-27.

22. Seki, Y, Yamamoto, H, Ngan, C Y, Yasui, M, Tomita, N, Kitani, K, et al. (2006). Construction of a novel DNA decoy that inhibits the oncogenic beta-catenin/T-cell factor pathway. Molecular cancer therapeutics 5: 985-994.

23. Cadigan, K M, and Waterman, M L (2012). TCF/LEFs and Wnt signaling in the nucleus. Cold Spring Harb Perspect Biol 4.

24. Krause, K, and Foitzik, K (2006). Biology of the hair follicle: the basics. Seminars in cutaneous medicine and surgery 25: 2-10.

25. Stenn, K S, and Paus, R (2001). Controls of hair follicle cycling. Physiological reviews 81: 449-494.

26. Paus, R, and Foitzik, K (2004). In search of the “hair cycle clock”: a guided tour. Differentiation; research in biological diversity 72: 489-511.

27. Liu, S, Zhang, H, and Duan, E (2013). Epidermal Development in Mammals: Key Regulators, Signals from Beneath, and Stem Cells. International Journal of Molecular Sciences 14: 10869-10895.

28. Collins, C A, Kretzschmar, K, and Watt, F M (2011). Reprogramming adult dermis to a neonatal state through epidermal activation of β-catenin. Development (Cambridge, England) 138: 5189-5199.

29. Van Mater, D, Kolligs, F T, Dlugosz, A A, and Fearon, E R (2003). Transient activation of beta-catenin signaling in cutaneous keratinocytes is sufficient to trigger the active growth phase of the hair cycle in mice. Genes & development 17: 1219-1224.

30. Orsulic, S, Huber, O, Aberle, H, Arnold, S, and Kemler, R (1999). E-cadherin binding prevents beta-catenin nuclear localization and beta-catenin/LEF -1-mediated transactivation. Journal of cell science 112 (Pt 8): 1237-1245.

31. Wu, C-I, Hoffman, J A, Shy, B R, Ford, E M, Fuchs, E, Nguyen, H, et al. (2012). Function of Wnt/β-catenin in counteracting Tcf3 repression through the Tc3-β-catenin interaction. Development (Cambridge, England) 139: 2118-2129.

32. Brannon, M, Gomperts, M, Sumoy, L, Moon, R T, and Kimelman, D (1997). A beta-catenin/XTcf-3 complex binds to the siamois promoter to regulate dorsal axis specification in Xenopus. Genes & development 11: 2359-2370.

33. Molenaar, M, van de Wetering, M, Oosterwegel, M, Peterson-Maduro, J, Godsave, S, Korinek, V, et al. (1996). XTcf-3 transcription factor mediates beta-catenin-induced axis formation in Xenopus embryos. Cell 86: 391-399.

34. Tian, X, Liu, Z, Niu, B, Zhang, J, Tan, T K, Lee, S R, et al. (2011). E-cadherin/beta-catenin complex and the epithelial barrier. J Biomed Biotechnol 2011: 567305.

35. Strauss, R, Hamerlik, P, Lieber, A, and Bartek, J (2012). Regulation of stem cell plasticity: mechanisms and relevance to tissue biology and cancer. Molecular therapy: the journal of the American Society of Gene Therapy 20: 887-897.

36. Chen, B, Dodge, M E, Tang, W, Lu, J, Ma, Z, Fan, C-W, et al. (2009). Small molecule-mediated disruption of Wnt-dependent signaling in tissue regeneration and cancer. Nature chemical biology 5: 100-107.

37. Jamora, C, DasGupta, R, Kocieniewski, P, and Fuchs, E (2003). Links between signal transduction, transcription and adhesion in epithelial bud development. Nature 422: 317-322.

38. Rivera-Gonzalez, G, Shook, B, and Horsley, V (2014). Adipocytes in Skin Health and Disease. Cold Spring Harbor Perspectives in Medicine 4: a015271.

39. Sumikawa, Y, Inui, S, Nakajima, T, and Itami, S (2014). Hair cycle control by leptin as a new anagen inducer. Experimental dermatology 23: 27-32.

40. Driskell, R R, Clavel, C, Rendl, M, and Watt, F M (2011). Hair follicle dermal papilla cells at a glance. Journal of cell science 124: 1179-1182.

41. Huelsken, J, Vogel, R, Erdmann, B, Cotsarelis, G, and Birchmeier, W (2001). β-Catenin Controls Hair Follicle Morphogenesis and Stem Cell Differentiation in the Skin. Cell 105: 533-545.

42. Alonso, L, and Fuchs, E (2006). The hair cycle. Journal of cell science 119: 391-393.

43. Blanpain, C, and Fuchs, E (2006). Epidermal Stem Cells of the Skin. Annual review of cell and developmental biology 22: 339-373.

44. Oshima, H, Rochat, A, Kedzia, C, Kobayashi, K, and Barrandon, Y (2001). Morphogenesis and renewal of hair follicles from adult multipotent stem cells. Cell 104: 233-245.

45. Taylor, G, Lehrer, M S, Jensen, P J, Sun, T T, and Lavker, R M (2000). Involvement of follicular stem cells in forming not only the follicle but also the epidermis. Cell 102: 451-461.

46. Lay, K, Kume, T, and Fuchs, E (2016). FOXC1 maintains the hair follicle stem cell niche and governs stem cell quiescence to preserve long-term tissue-regenerating potential. Proceedings of the National Academy of Sciences of the United States of America 113: E1506-E1515.

47. Geyfman, M, Plikus, M V, Treffeisen, E, Andersen, B, and Paus, R (2015). Resting no more: re-defining telogen, the maintenance stage of the hair growth cycle. Biological reviews of the Cambridge Philosophical Society 90: 1179-1196.

48. Paus, R, and Cotsarelis, G (1999). The biology of hair follicles. The New England journal of medicine 341: 491-497.

49. Porter, R M (2003). Mouse models for human hair loss disorders. Journal of anatomy 202: 125-131.

50. Muller-Rover, S, Handjiski, B, van der Veen, C, Eichmuller, S, Foitzik, K, McKay, I A, et al. (2001). A comprehensive guide for the accurate classification of murine hair follicles in distinct hair cycle stages. The Journal of investigative dermatology 117: 3-15.

51. Garza, L A, Liu, Y, Yang, Z, Alagesan, B, Lawson, J A, Norberg, S M, et al. (2012). Prostaglandin D2 inhibits hair growth and is elevated in bald scalp of men with androgenetic alopecia. Science translational medicine 4: 126ra134.

52. Halloy, J, Bernard, B A, Loussouarn, G, and Goldbeter, A (2000). Modeling the dynamics of human hair cycles by a follicular automaton. Proc Natl Acad Sci U S A 97: 8328-8333.

53. Ghatak, S, Li, J, Chan, Y C, Gnyawali, S C, Steen, E, Yung, B C, et al. (2016). AntihypoxamiR functionalized gramicidin lipid nanoparticles rescue against ischemic memory improving cutaneous wound healing. Nanomedicine 12: 1827-1831.

54. Patel, V, Rink, C, Gordillo, G M, Khanna, S, Gnyawali, U, Roy, S, et al. (2012). Oral tocotrienols are transported to human tissues and delay the progression of the model for end-stage liver disease score in patients. J Nutr 142: 513-519.

55. Sen, C K, Khanna, S, Babior, B M, Hunt, T K, Ellison, E C, and Roy, S (2002). Oxidant-induced vascular endothelial growth factor expression in human keratinocytes and cutaneous wound healing. The Journal of biological chemistry 277: 33284-33290.

56. Chan, Y C, Khanna, S, Roy, S, and Sen, C K (2011). miR-200b targets Ets-1 and is down-regulated by hypoxia to induce angiogenic response of endothelial cells. The Journal of biological chemistry 286: 2047-2056.

57. Ghatak, S, Chan, Y C, Khanna, S, Banerjee, J, Weist, J, Roy, S, et al. (2015). Barrier Function of the Repaired Skin Is Disrupted Following Arrest of Dicer in Keratinocytes. Molecular therapy: the journal of the American Society of Gene Therapy 23: 1201-1210.

58. Gordillo, G M, Biswas, A, Khanna, S, Spieldenner, J M, Pan, X, and Sen, C K (2016). Multidrug Resistance-associated Protein-1 (MRP-1)-dependent Glutathione Disulfide (GSSG) Efflux as a Critical Survival Factor for Oxidant-enriched Tumorigenic Endothelial Cells. The Journal of biological chemistry 291: 10089-10103.

59. Kuhn, D E, Roy, S, Radtke, J, Khanna, S, and Sen, C K (2007). Laser microdissection and capture of pure cardiomyocytes and fibroblasts from infarcted heart regions: perceived hyperoxia induces p21 in peri-infarct myocytes. Am J Physiol Heart Circ Physiol 292: H1245-1253.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of increasing skin stem cells in a subject, comprising:
administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and delta-tocotrienol; and
a pharmaceutically acceptable excipient;
wherein the tocotrienol composition is administered at a dose of about 1 mg/cm² to about 10 mg/cm²; and
wherein the tocotrienol composition is administered in an amount sufficient to increase skin stem cells in the subject.

2. The method of claim 1, wherein the increase in skin stem cells is determined by measuring the expression of a stem cell marker.

3. The method of claim 2, wherein the stem cell marker is selected from Oct4, Sox9, K17, K15, or LGR-6.

4. The method of claim 2, wherein the stem cell marker is selected from Oct4, Sox9 or LGR-6.

5. The method of claim 1, wherein the tocotrienol composition further comprises an additional dermatological agent.

6. The method of claim 5, wherein the additional dermatological agent is an antibiotic or antifungal.

7. The method of claim 1, wherein the tocotrienol composition further comprises alpha-tocopherol.

8. The method of claim 1, wherein the tocotrienol composition comprises tocopherol, by weight percent of total, less than a percent selected from the group consisting of: 50%; 40%; 30%; 20%; 15%; 10%; 5%; and 1%.

9. The method of claim 7, wherein the tocotrienol composition comprises 17-34% alpha-tocotrienol, 2-4% beta-tocotrienol; 27-54% gamma-tocotrienol, 8-23% delta tocotrienol, and 14-32% alpha tocopherol, by weight of those five ingredients.

10. The method of claim 1, wherein the tocotrienol composition is derived from palm oil.

11. The method of claim 1, wherein the tocotrienol composition is administered at a dose of about 5 mg/cm².

12. A method for the prevention or treatment of a skin disorder, comprising:
administering to the subject an effective amount of a topical tocotrienol composition three times per week, wherein the topical tocotrienol composition comprises:
at least one tocotrienol selected from the group consisting of: alpha-tocotrienol; beta-tocotrienol; gamma-tocotrienol; and delta-tocotrienol; and
a pharmaceutically acceptable excipient;
wherein the tocotrienol composition is administered at a dose of about 1 mg/cm² to about 10 mg/cm²; and
wherein the tocotrienol composition is administered in an amount sufficient to prevent or treat the skin disorder in the subject.

13. The method of claim 12, wherein the tocotrienol composition further comprises an additional dermatological agent.

14. The method of claim 13, wherein the additional dermatological agent is an antibiotic or antifungal.

15. The method of claim 12, wherein the tocotrienol composition further comprises alpha-tocopherol.

16. The method of claim 12, wherein the tocotrienol composition comprises tocopherol, by weight percent of total, less than a percent selected from the group consisting of: 50%; 40%; 30%; 20%; 15%; 10%; 5%; and 1%.

17. The method of claim 15, wherein the tocotrienol composition comprises 17-34% alpha-tocotrienol, 2-4% beta-tocotrienol, 27-54% gamma-tocotrienol, 8-23% delta tocotrienol, and 14-32% alpha tocopherol, by weight of those five ingredients.

18. The method of claim 12, wherein the tocotrienol composition is derived from palm oil.

19. The method of claim 12, wherein the tocotrienol composition is administered at a dose of about 5 mg/cm².

20. The method of claim 12, wherein the skin disorder is selected from skin injury, UV exposure related injury, sunburn, melanoma, wrinkles, psoriasis, severe dryness, itchiness, microbial infections, or fungal infections.
